(19) United States
(12) Patent Application Publication
(10) Pub. No.: US 2026/0199224 A1
(43) Pub. Date: Jul. 16, 2026

(54) Compositions comprising Sanguisorba officinalis root extract and uses thereof

(71) Applicants: Clariant International Ltd, Muttenz (CH); Plant Advanced Technologies PAT S.A, Vandoeuvre-lès-Nancy (FR)

(72) Inventors: Mathilde FRECHET, Toulouse (FR); Hanane CHAJRA, Tournefeuille (FR); Sandrine DELAUNOIS, Toulouse (FR); Benoit MIGNARD, Toulouse (FR); Thomas REGNAULT, Nancy (FR); 6) Aleksander SALWINSKI, Nancy (FR); Natacha ROMBAUT, Nancy (FR)

(21) Appl. No.: 19/135,087

(22) PCT Filed: Nov. 24, 2023
(86) PCT No.: PCT/EP2023/083005

§ 371 Date: Jun. 3, 2025

(30) Foreign Application Priority Data
Dec. 5, 2022 (EP) 22315304.0

(51) Int. Cl. A61K 8/9789; A61K 8/63; A61K 31/56; A61K 36/739; A61P 17/00; A61Q 5/00; A61Q 17/04; A61Q 19/00; A61Q 19/02; A61Q 19/08

(52) U.S. Cl. CPC A61K 8/9789; A61K 8/63; A61K 31/56; A61K 36/739; A61P 17/00; A61Q 5/00; A61Q 17/04; A61Q 19/00; A61Q 19/007; A61Q 19/02; A61Q 19/08; A61K 2236/11; A61K 2236/33; A61K 2800/522; A61K 2800/591; A61K 2800/74; A61K 2800/805

(57) ABSTRACT

Compositions comprising Sanguisorba officinalis root extract and uses thereof. The present invention relates to a root extract of Sanguisorba officinalis and use thereof as an active ingredient for skin care and/or scalp care.

### FIELD OF THE INVENTION

The present invention refers to a root extract of the plant Sanguisorba officinalis for promoting the light-related human being mood. The present invention also relates to a root extract of Sanguisorba officinalis enriched in feruloylated tormentic acid derivatives and a method for preparing such extract. Furthermore, the present invention refers to a composition comprising such root extract and to the use of such root extract as an active ingredient for skin care and/or scalp care for promoting the light-related human being mood.

### BACKGROUND OF THE INVENTION

Sunlight is essential to many organisms, including human being, and exerts several powerfull biological effects beyond visual responses. It includes circadian rhythm regulation and brain activity, leading to modulations of sleep and learning. Moreover, sunlight directly influence human being mood without causing circadian arrhythmicity or sleep disruptions (LeGates et al., Nature Reviews Neuroscience, Vol. 15, pages 443-454, 2014). It is well documented that lack of light is linked to depressive symptoms and cognitive dysfunction. Retina is the key organ involved in light detection, and opsins are key phototransducing molecules found in it. Being the organ most exposed to light, skin expresses also photoreceptors called opsins. Besides retina, opsins are expressed in epidermis, in dermis and in hair follicle. Differents opsins have been described, each being responsive to different wavelenghth of light. In the eye, one of the most abundant opsins is peropsin (RRH) which is also expressed in human skin. The biological role of opsins in skin physiology are not yet very known but some have been linked to skin differentiation, circadian rhythm, dermal matrix remodeling through matrix metalloproteinases (MMPs) modulation or wound healing stimulation (Suh et al., Photodermatol Photoimmunol Photomed, Vol. 36 (5), pages 329-338, 2020). The skin ability to sense and transduct light independently to eye involvement through its retina have been done on human by illuminating the skin. It has been demonstrated that a lighting behind the knee influenced circadian rhythm or that a UV-A exposure done with opaque goggles lead to an increase of serum serotonin and a feeling of being more balanced and less nervous.

Serotonin, a body's natural mood booster, is an important mediator of bidirectional interactions between the neuroendocrine system and the skin. Human skin has a serotonergic system capable of generating serotonin. This places the skin in a central place in the link between light and good mood, in which serotonin is of great importance. Serotonin is also the precursor of melatonin, which is the “sleep hormone”.

Melatonin is a neurohormone entrained by the light/dark cycle. Light may either suppress or synchronize melatonin production according to the light schedule and melatonin may stabilise and may strengthen coupling of circadian rhythms. Daylight exposure has been shown to stimulate melatonin level in the morning without acting on melatonin level in the evening while improving sleep on human. Melatonin is also known to be synthesized and metabolized in the skin, where it plays several roles. Some of its functions are related to the antioxidant properties and includes protection against UV rays and X-rays. Particularly, melatonin is a strong protector against UV radiations, fundamentally against UV-B radiations. Furthermore, melatonin also activates the antioxidant cascade reducing free radicals and DNA repair systems in the skin. Finally, melatonin enhances the skin barrier through the stimulation of the expression of involucrin keratin-10 and keratin-14, and promotes skin wound healing as well.

Another sunlight related beneficial effects may be the production of the essential nutrient vitamin D, often called the “sunshine” vitamin. The skin is the main source of vitamin D of the human body following sun exposure. Unfortunately, roughly 75% of the worldwide population are deficient in vitamin D due to the significant time spent indoors. Upon the absorption of UV-B, the precursor 7-dehydrocholesterol (7-DHC) is transformed to vitamin D3 in the skin, a process accelerated by thermal energy. These reactions are non-enzymatic and dependent on the UVB dose and the temperature. 7-DHC in keratinocytes and dermal fibroblasts is converted to previtamin D. Vitamin D is enzymatically hydroxylated to 25-hydroxyvitamin D (25(OH)D) then to its biologically active metabolite 1,25(OH)2D. Cutaneously synthesized vitamin D may be released from the plasma membrane and enters the systemic circulation bound to vitamin D-binding protein (DBP). Vitamin D produced in the skin supplies more than 90% of the vitamin D body's requirement. Vitamin D status impacts cognitive, behavioral, and mood disorders. In addition, people with vitamin D insufficiency are more likely to have any kind of sleep disorder, poorer sleep quality, shorter sleep duration, and/or excessive daytime sleepiness.

The skin itself is capable of responding to the active metabolites of vitamin D3. The main genomic effects and biological responses of vitamin D3 metabolites in the skin are mediated through their binding to the nuclear vitamin D receptor (VDR). Indeed, the skin expresses the VDR and serves as a site for the action of vitamin D. VDR activated by classical 1,25(OH)2D3 induces rapid response signaling through a non-genomic, membrane-associated mechanism based on an alternative ligand-binding site or through action on 1,25D3-MARRS receptor. Vitamin D can exert several different effects on the main skin cells (keratinocytes and fibroblasts) and immune cells through the activation of nuclear VDR. Vitamin D also plays a pivotal role in skin homeostasis contributing to its barrier function and favors skin differentiation. Its deficiency has been linked to many proliferative and inflammatory cutaneous disorders. Moreover, as an essential part of a functioning immune system, active forms of vitamin D may modulate the cutaneous immunity, exerts antioxidant, anti fibrotic and anti-inflammatory properties.

Sunlight also is also beneficial for the maintenance of a balanced circadian rhythm, which is fundamental in regulating a wide range of cellular, metabolic, physiological, and behavioral activities in mammals. Master clock is greatly influenced by light but also by the environment. Focusing on light, its effects on the phase of the circadian clock depends on the timing of light exposure. In this regard, several key circadian genes have been identified in the skin.

Thanks to these proven light beneficial effects, light therapies or photobiomodulation are used for both the treatment of mood disorders and the treatment of various dermatological conditions, such as psoriasis, atopic dermatitis, hair regrowth, wound healing, and tissue regeneration.

On the other side, the sunlight also brings deleterious effects on skin called photodamages such as hyperpigmentation, melanoma due to DNA damages, premature aging, weaken mechanical properties, namely loss of elasticity and firmness due to the degradation of the extracellular matrix (ECM) components, dehydration, and alteration of skin complexion. It has been showed that broad protection against the entire solar range of UV-B, UV-A, visible light, and short infrared is required to prevent sunlight-related skin damages.

Carbonylated proteins (CPs) are synthesized by reactions between amino groups in proteins and reactive aldehyde compounds yielded from lipid peroxidation initiated by reactive oxygen species. In the skin, CPs are detected in a higher frequency at sun-exposed sites of the skin in elderly subjects [1]. CPs are also detected in the stratum corneum (SC) collected from the skin in the winter season in spite of the weak radiation energy of sunlight at that time [2]. CPs have been reported to be correlated with decrease skin water content and transepidermal water loss (TEWL), alterations of the dermal matrix, increase of darkness around facial poresand a change of the skin color of yellow-dark which alter skin tone (Masaki et al., J. Dermatol Science, Vol. 84, 1: pages 05-16 [01-04], 2016).

Sanguisorba officinalis, also called the great burnet or sanguisorbe officinale, is a perennial herbaceous plant of the Rosaceae family and is present in the northern hemisphere, in Europe, in Asia and in North America.

Root extracts of Sanguisorba officinalis are known to contain metabolites such as ellagitanins, flavonoids and polyphenols. These extracts have reportedely antiviral, antibacterial, haemostatic, anti-inflammatory and anti-cancer activities, whose ziyuglycosides I and II and sanguiin H-6 only partly explaining these activities (Jang et al., A Review, The American Journal of Chinese Medicine, Vol. 46, No. 2, 1-20, 2018).

Root extract of Sanguisorba officinalis further contains euscaphic acid and/or tormentic acid(s) (Seongdae et al., Molecules, 23, 3001, 2018), ferulic acid or its esters. Ferulic acid exhibits antioxidant and anti-tyrosinase activities.

Tormentic acid belongs to triterpenoids compounds having six isoprene units. It is known to have an anticancer, anti-atherosclerosis, anti-inflammatory, anti-diabetic, antimicrobial, cardio- and neuroprotective properties. Tormentic acid can be broadly found in many plants, but it also exists in various derivative forms which are more specific to certain families of plants and to some tissues. If euscaphic acid or the glycosylated forms of tormentic acid are the most common ones, these forms hold original activities that often differ from the basic form (tormentic acid).

Feruloylated derivatives of tormentic acid are rare natural molecules which may be for interest in cosmetic field. In particular, 3-O-trans-feruloyl tormentic acid has only been found in extract of the plants Vitex rotundifolia and Vitex trifolia and 3-O-trans-feruloyl euscaphic acid in the leaf extract of Eriobotrya japonica. Another compound, Oryzanol, with a close structure from ferulic acid coupled with a triterpene, but not tormentic acid, did not demonstrate any toxicity in mice suggesting that these molecules may be for interest in cosmetic.

It has been discovered that the roots of Sanguisorba officinalis may contain ferulic acid coupled with tormentic acid via ester bond unlike the aerial parts. Therefore, accessing to the roots of Sanguisorba officinalis is key for recovering these compounds of interest. However, such operation involves the destruction of all or part of the plant, which is not desirable with regards to the Nagoya Protocol and the International Standard (ISO 26000) guidelines in order to control the access to the biodiversity and for ensuring a sustainable development and social responsibility.

The use of the extracts of Sanguisorba officinalis is known in cosmetics. For example, CN-B 105193680 and KR-A 20190003011 disclose extract of the root of Sanguisorba officinalis for promoting the skin whitening property. According to CN-B 105193680, the root extract is obtained by using a resin fractionation step during which many active components, including feruloylated tormentic acid derivatives, are certainly adsorbed in the resin. EP-B1 0993826 discloses a root extract of Sanguisorba officinalis for stimulating the melanogenesis in the skin. JP-B 3449967 discloses a root extract of Sanguisorba officinalis for stimulating the collagen synthesis after UV-B exposure. WO-A1 2018000060 discloses a root extract of Sanguisorba officinalis as a sebum regulator agent for greasy skin. EP-A2 1051965 discloses a root extract of Sanguisorba officinalis for improving the ceramide production and, hence, enhancing the skin moisturizing effect.

Ziyuglycoside I, a triterpenoid saponin, isolated from the ethanolic extract of the root of Sanguisorba officinalis has been reported to exhibit activities such as anti-inflammatory, and anti-wrinkles (Young Heui KIM et al., Bioscience, Biotechnology and Biochemistry, 72:2, 303-311, 2014, DOI: 10.1271/bbb.70268).

As a large proportion of the worldwide population are spending most of their time indoors, there is an unmet need for a product of natural origin that may be used to mimic, at least partly, the beneficial effects which may be brought by sunlight on the body, preferably on the skin, even in the absence of and/or in weak sunlight exposure conditions. Furthermore, it has been actually observed that more and more of consumers of cosmetic products are looking for products of natural origins, which are enable to fit with the two criterion. The first criteria relates to the visual effects observed after application of these products. The second criteria is more related to the emotional aspect, in particular the well-being that users could feel after application of these products.

Accordingly, there is an unmet need in providing products of natural origins, namely a root extract of Sanguisorba officinalis capable to bring visual benefical effects on the body, in particular in the skin and/or the scalp, and also to exhibit a well-being sensation in a subject.

Sunlight may also bring well-known deleterious effects on the skin. Therefore, there is also an unmet need for a product of natural origin for skin care, preferably capable to prevent light-related skin damages, and hence, to delay the signs of photoaging.

It has surprisingly found that a root extract of Sanguisorba officinalis may be capable to mimic the sunlight related effects on the human body, in particular in the skin, even in the absence of and/or in weak light exposure conditions. In this regard, a root extract of Sanguisorba officinalis may be used, for example in a light-like therapy, for improving the light-related human being mood, for obtaining the associated light-beneficial effects on the body, and hence the relaxation sensations, even in the absence of and/or in weak light exposure conditions.

It has also been surprisingly found that a root extract of Sanguisorba officinalis may be enriched in some components of interest which may natively be present in a low amount. Indeed, a root of Sanguisorba officinalis may be enriched in feruloylated derivatives of tormentic acid, for example, by culturing said plant under certain conditions without compromising the overall plant development. The said enriched root extract may be advantageously used for skin care and/or scalp care.

### SUMMARY OF THE INVENTION

An aspect of the present invention refers to a root extract of Sanguisorba officinalis comprising:
a tormentic acid representing at least 1% by weight relative to the total weight of the dry extract,
a feruloylated derivative of tormentic acid having the general formula (I)

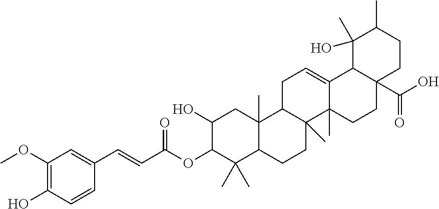

and
a feruloylated derivative of deoxy-tormentic acid having the general formula (II)

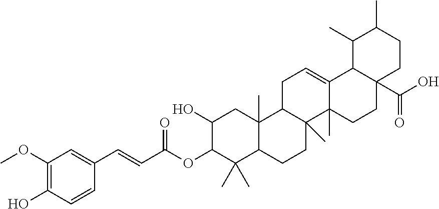

The present invention further refers to a method for preparing a root extract of the plant Sanguisorba officinalis, said method comprising the following steps:
a) cultivating Sanguisorba officinalis under soilless conditions, in particular aeroponically,
b) stimulating the roots of said plant,
c) solid/liquid extraction by maceration of the roots obtained in step b),
d) recovering the extract obtained in step c), and
e) optionally, diluting and/or clarifying the extract recovered in step d) by successive filtrations.

The present invention further refers to a cosmetic or dermatological or nutraceutical composition comprising a root extract of Sanguisorba officinalis as defined above, and optionally one or more excipients, which are preferably cosmetically or dermatologically or nutraceutically acceptable.

The present invention further refers to the (cosmetic) use of a root extract of Sanguisorba officinalis as defined above as an active ingredient for skin care and/or scalp care.

The present invention also refers to a non-therapeutic method for preventing or delaying the appearance of skin aging effects, for promoting the light-related human being mood, the method comprising the application on at least a part of the body a root extract of Sanguisorba officinalis as defined above.

Another aspect of the present invention refers to the (cosmetic) use of a root extract of Sanguisorba officinalis for promoting the light-related human being mood.

All documents cited or referenced herein (“herein cited documents”) together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### DESCRIPTION OF FIGURES

FIGS. 1A and 1B show the stimulation of the well-being hormones production in skin explants treated with a root extract of Sanguisorba officinalis according to an aspect of the invention. FIG. 1A shows the concentration of serotonin released by skin cells in media culture after 3 days of treatment. FIG. 1B shows the concentration of melatonin released by skin cells in media culture after 5 days of treatment.

FIGS. 2A and 2B show a clinical evaluations related to the improvement of well-being sensation on volunteers topically treated by a root extract of Sanguisorba officinalis according to an aspect of the invention. FIG. 2A demonstrated a relaxation improvement between day 1 and 5 days post-treatment. FIG. 2B demontrated a relaxation improvement at 7 days post-treatment.

FIG. 3 shows an auto-evaluation questionnaire related to the well-being effect on a panel of 36 volunteers topically treated by a root extract of Sanguisorba officinalis according to an aspect of the invention.

FIGS. 4A and 4B show the regulation of vitamin D downstream biological pathway in the skin by a root extract of Sanguisorba officinalis according to an aspect of the invention. FIG. 4A shows the regulation of vitamin D receptor. FIG. 4B shows the regulation of vitamin D binding protein.

FIG. 5 shows the induction and potentialization of light transduction signal on the skin explants treated with a tormentic acid and a feruloylated tormentic acid derivative isolated from a root extract of Sanguisorba officinalis according to the an aspect of the invention.

FIG. 6 shows an improvement of the skin elasticity observed during a clinical study conducted on volunteers topically treated by a root extract of Sanguisorba officinalis according to an aspect of the invention.

FIG. 7 shows an improvement of skin complexion observed during a clinical study performed on volunteers topically treated by a root extract of Sanguisorba officinalis according to an aspect of the invention

FIG. 8 shows the carbonylated proteins content in corneocytes of volunteers treated with a root extract of Sanguisorba officinalis according to an aspect of the invention after 28 days of application.

The elements of the present invention will be described in more detail. These elements are listed with specific embodiments; however, they may be combined in any manner and in any number to create additional embodiments. The variously described examples and embodiments should not be construed to limit the present invention to only the explicitly described embodiments. This description should be understood to support and encompass embodiments which combine the explicitly described embodiments with any number of the disclosed elements. Furthermore, any permutations and combinations of all described elements in this application should be considered disclosed by the description of the present application unless the context indicates otherwise.

Throughout this specification and the claims, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by the context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”), provided herein is intended merely to better illustrate the invention and does not pose a limitation on the scope of the invention otherwise claimed. No language in the specification should be construed as indicating any non-claimed element essential to the practice of the invention.

Unless defined otherwise, all technical and scientific terms used herein have the same meaning as commonly understood by a skilled person. Although the methods and materials described herein are preferred, other methods and materials similar or equivalent to those described herein can be used in the practice or testing of the present invention as well.

### DETAILED DESCRIPTION OF THE INVENTION

An aspect of the present invention relates to a root extract of Sanguisorba officinalis comprising:
a tormentic acid representing at least 1% by weight relative to the total weight of the dry extract,
a feruloylated derivative of tormentic acid having the general formula (I),

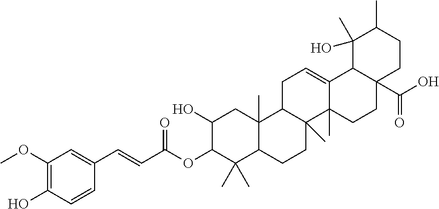

and
a feruloylated derivative of deoxy-tormentic acid having the general formula (II)

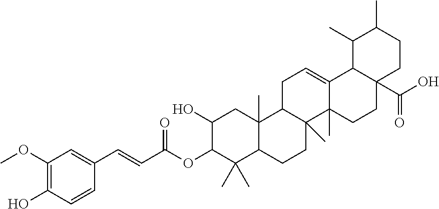

According to an embodiment, the tormentic acid represents 1 to 10% by weight, preferably 1.2 to 8% by weight, more preferably 1.5 to 7% by weight, in particular 1.8 to 5% by weight relative to the total weight of the dry extract.

Acording to an embodiment, the feruloylated derivative of tormentic acid represents at least 0.1% by weight relative to the total weight of the dry extract. Advantageously, the feruloylated derivative of tormentic acid represents 0.1 to 5% by weight, preferably 0.2 to 4.5% by weight, more preferably 0.3 to 4% by weight, even more preferably 0.4 to 3.5% by weight, in particular 0.5 to 3% by weight, relative to the total weight of the dry extract.

According to an embodiment, the feruloylated derivative of deoxy-tormentic acid represents at least 0.05% by weight, relative to the total weight of the dry extract. Advantageously, the feruloylated derivative of deoxy-tormentic acid represents 0.05 to 3% by weight, preferably 0.06 to 2.8% by weight, more preferably 0.07 to 2.6% by weight, even more preferably 0.08 to 2.4% by weight, most preferably 0.09 to 2.2%, in particular 0.1 to 2% by weight by weight, relative to the total weight of the dry extract.

In a preferred embodiment, the feruloylated derivative of tormentic acid represents at least 0.1% by weight and the feruloylated derivative of deoxy-tormentic acid represents at least 0.05% by weight, all weight being relative to the total weight of the dry extract.

According to an embodiment, the tormentic acid, the feruloylated derivative of tormentic acid and the feruloylated derivative of deoxy-tormentic acid represent together at least 1.15% by weight relative tot he total weight of the dry extract. Advantageously, the tormentic acid, the feruloylated derivative of tormentic acid and the feruloylated derivative of deoxy-tormentic acid represent together 1.15 to 18% by weight, preferably 1.30 to 15% by weight, even more preferably 1.40 to 12% by weight, in particular 1.60 to 10% by weight, relative to the total weight of the dry extract.

According to an embodiment, the root extract may further contain one or more ellagitanins.

According to an embodiment, the root extract may be in liquid form and may comprise a solvent selected from water, lower alcohols, glycols or a mixture thereof, and dicaprylyl ether. The lower alcohols may be preferably selected from methanol and ethanol. The glycols may be preferably selected from dipropylene glycol, 1,2-propanediol, 1,3-propanediol, 1,3-butanediol, pentylene glycol and glycerol. Preferably, the solvent may be selected from 1,3-propanediol, 1,3-butanediol and glycerol. More preferably, the solvent is 1,3-propanediol. Particularly preferably, the solvent is bio-based 1,3-propanediol.

Accordingly, a liquid root extract may correspond to a raw liquid extract obtained after the step c) of solid/liquid extraction by root (optionally stimulated) maceration of the roots and also after the step d). A “maceration solvent” is thus a solvent used for obtaining the root extract. Such solvents need to be selected from particular solvents in order to reach the desired content of feruloylated derivative of tormentic acid and feruloylated derivative of deoxy-tormentic acid.

According to an embodiment, said root extrat may be in a solid or in a sticky form after a further step e) of drying the root extract in a liquid form, the drying being conducted according to any method known in the art, for example for placing the root extract in liquid form in a hot and dry atmosphere to evaporate the maceration solvent.

The root extract in solid or sticky form may further be diluted in a dilution solvent to obtain another type of root extract in liquid form. A “dilution solvent” is thus a solvent used for diluting an already obtained root extract in solid or sticky form. Advantageously, such root extract comprises a tormentic acid, the feruloylated derivative of tormentic acid and the feruloylated derivative of deoxy-tormentic acid. The dilution solvent may notably be selected from alcohols, glycols, ethyl lactate, isopropyl myristate, triglycerides, tri-ethyl citrate, dicaprylyl ether, glyceryl isostearate, glyceryl stearate, ethyl acetate, vegetable oils or a mixture thereof. The alcohols are preferably selected from methanol and ethanol. Indeed, while specific solvents need to be used as maceration solvents in order to reach the desired content of feruloylated derivative of tormentic acid, and the feruloylated derivative of deoxy-tormentic acid, other solvents may be used for diluting an already obtained root extract in solid or sticky form before use of the extract. In particular, the dilution solvent does not need to be the same as the maceration solvent used for the obtention of the extract of the present invention. Accordignly, the dilution solvents may preferably be selected from methanol, ethanol, 1,3-propanediol, pentylene glycol, glycerol, dicaprylyl ether, particularly bio-based dicaprylyl ether.

Depending on if the root extract is in liquid form, a raw liquid root extract in maceration solvent or a dried root extract is diluted in a dilution solvent, a root extract in liquid form may comprise a maceration solvent as disclosed above or a dilution solvent as disclosed above.

By “tormentic acid” is meant tormentic acid itself as well as its stereoisomers.

By “feruloylated derivative of tormentic acid” is meant a compound corresponding to the molecular formula: C40H56O8 and having the following general formula (I).

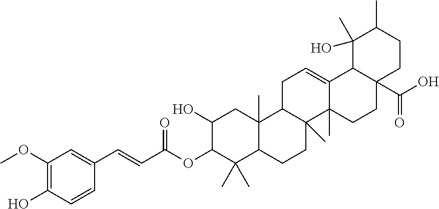

Formula (I): feruloylated derivative of tormentic acid

By “feruloylated derivative of deoxy-tormentic acid” is meant a compound corresponding to the molecular formula: C40H56O7 and having the following general formula (II).

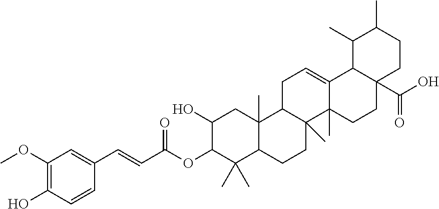

Formula (II): feruloylated derivative of deoxy-tormentic acid

By “root enriched in feruloylated derivatives of tormentic acid” and “enriched root” are meant a root extract of said plant containing a higher amount of at least one feruloylated derivative of tormentic acid (formula (I) or formula (II)), in comparison to a corresponding root extract of the same plant that can be found in nature.

By “feruloylated derivatives of tormentic acid” is meant feruloylated derivative of tormentic acid and feruloylated derivative of deoxy-tormentic acid.

Preferably, for obtaining the root extract of Sanguisorba officinalis according to an aspect of the present invention, when the roots are considered sufficiently developed, they are contacted with an extraction solvent by immersion or preferably by maceration, and then said extraction solvent is recovered and treated to extract secondary metabolites, including the feruloylated derivatives of tormentic acid, therefrom which have been released by the roots. This preferred method is adapted from the method developed by the company named Plant Advanced Technologies (PAT) as “PAT Plantes à Traire®” and described in the international application WO 01/33942. The teaching of this document is incorporated by reference in the specification of the present invention.

Accordingly, the present invention further refers to a method for preparing a root extract of the plant Sanguisorba officinalis according to an aspect of the invention, said method comprising the following steps:
a) cultivating Sanguisorba officinalis under soilless conditions, in particular aeroponically,
b) stimulating the roots of said plant,
c) solid/liquid extraction by maceration of the roots obtained in step b),
d) recovering the extract obtained in step c), and
e) optionally, diluting and/or clarifying the extract recovered in step d) by successive filtrations.

In an embodiment, the root extract of Sanguisorba officinalis according to an aspect of the invention may be obtained by the method described above.

By “cultivation of plants under soilless conditions” is meant any mode of cultivation in which the development of plant roots does not take place in the soil. More precisely, soilless cultivation is a cultivation-type in which the roots of the plants grow in a reconstituted medium, detached from the soil. This cultivation medium is regularly irrigated with well-known nutrient solutions suitable for the cultivated plant.

Various soilless cultivation techniques are known, such as substrate-free systems which require an oxygen-enriched nutrient solution, and substrate-based systems. Among the substrate-free systems are aquaculture, for which the nutrient solution is non-circulating and is contained in a cultivation tank, the Nutrient Film Technique (NFT) for which the nutrient solution becomes enriched in dissolved oxygen in the course of its movement by exchange with air, and aeroponics, for which the roots of the plants are not in contact neither with a solid medium, nor with a liquid medium. Indeed, roots are fed by a nutrient mist obtained by misting the nutrient solution in a closed medium. Among the substrate-based systems is subirrigation, in which the nutrient solution penetrates the substrate via its lower part, and percolation, in which the nutrient solution is distributed by discontinuous irrigation at the upper surface of the system and then percolates to the bottom of the substrate. The mineral or organic substrate is neutral and inert, like sand, clay or rock wool, for example. This substrate may also be of synthetic origin.

The terms “aeroponic plant cultivation” and “aeroponically” refer to a mode of soilless cultivation for which the roots of the plants are not in permanent contact neither with a solid medium nor with a liquid nutrient medium.

The term “nutrient solution” refers to a solution comprising essential mineral salts (nitrogen—N, phosphorus—P, potassium—K), in an optimal amount and in an optimal ratio each other to obtain maximum root growth and a maximum production of secondary metabolites including feruloylated derivatives of tormentic acid, having formula I and/or formula II.

The term “stimulating nutrient solution” refers to a nitrogen-deficient solution comprising essential mineral salts (nitrogen—N, phosphorus—P, potassium—K), in optimal amount to obtain maximum root growth and a maximum production of secondary metabolites including feruloylated derivatives of tormentic acid, having formula I and formula II.

According to a preferred embodiment, the plant may be fed with a nutrient solution mist obtained by misting, via a mister, of the nutrient solution in a closed medium.

According to an embodiment, the plant may be placed on trays with the aerial part of the plant above the tray and the root part below, the trays are placed on tables forming a retention area to collect the excess of a liquid diffused toward the plants, and the trays are transferred onto the tables at various stations. Teachings about this technology suitable for the aeroponic plant culture is more detailed in the international application WO2018054704A1 which is also incorporated by reference in the specification of the present invention.

In a preferred embodiment, during step a), the aeroponically cultivated Sanguisorba officinalis is fed by spraying the roots with a nutrient solution of essential mineral salts (nitrogen—N, phosphorus—P, potassium—K), to obtain maximum root growth and a maximum concentration of the susmentioned secondary metabolites, without impairing the survival of the plants. By means of his general knowledge, a skilled person knows how to adapt the proportions and concentrations of the various mineral salts to optimize the plant growth and particularly the root growth. The mineral salt concentrations of the nutrient solutions are, in this case, typically within a low electrical conductivity range advantageously extending between 0.4 to 1.6 mS/cm, preferably between 0.8 to 1.2 mS/cm, to promote a greater diversity of secondary metabolites, including tormentic acid and feruloylated derivatives of tormentic acid, within the root extract.

Advantageously, the above aeroponic culture conditions allow the obtention of some secondary metabolites, including feruloylated derivatives of tormentic acid, in amount superior compared to the amount of these same compounds obtained in soil-culture.

In a preferred embodiment, the method may comprise a stimulation step of the roots. In this case, the step b) of root stimulation of said plant comprises a step of placing the roots in contact with a nitrogen-deficient stimulating nutrient solution, which may be a solution comprising a nitrogen proportion less than the nitrogen proportion usually considered as optimal for plant growth and in particular root growth. Advantageously the nutrient solution may contain less than 15% of nitrogen, and more advantageously not comprising any nitrogen, steps a) and b) being sequential or simultaneous.

In a preferred embodiment, the roots stimulation of said plant in step b) comprises a step of contacting the roots with a nitrogen-deficient nutrient solution. Placing the plants in contact with a nitrogen-deficient nutrient solution causes a “nitrogen stress” which is responsible for the stimulation of the production of secondary metabolites, particularly feruloylated derivatives of tormentic acid.

In a particular embodiment, said nitrogen-deficient stimulating nutrient solution is a solution comprising typically less than 15% of nitrogen, preferably less than 10% of nitrogen, advantageously less than 8%, more advantageously less than 6%, less than 5%, less than 4%, less than 3%, less than 2%, less than 1% of nitrogen and even more advantageously 0% of nitrogen.

In an embodiment, the root stimulation step b) allows to significantly increase the content of secondary metabolites, particularly feruloylated derivatives of tormentic acid, in the roots and thus to promote the flow of said metabolites going from the roots into the solvent chosen for the extraction, and to do so without total loss of viability of the plant so that it can be returned for culturing and then reused. In other words, the plant stimulation step promotes the biosynthesis of the susmentioned secondary metabolites.

According to an embodiment, the root stimulation in step b) may be performed by feeding the roots with a nitrogen-deficient N/P/K stimulating nutrient solution vaporized or misted onto the roots.

According to an embodiment, the step b) may be performed by spraying or macerating the roots with an N/P/K nutrient solution comprising less than 6% nitrogen, more preferably less than 3%, said solution preferably being vaporized or misted onto the roots.

According to an embodiment, the step b) of stimulating the roots by feeding the roots with a nitrogen-deficient N/P/K nutrient solution vaporized onto the said roots is advantageously performed for a time of between 1 week and 8 weeks, more particularly between 1 week and 3 weeks, preferably 2 weeks.

According to an embodiment, during step b), the mineral salt concentrations of the nutrient solutions may be within a low electrical conductivity range advantageously extending between 0.4 to 1.2 mS/cm, preferably between 0.6 to 1.0 mS/cm, to promote a greater diversity of the secondary metabolites, including feruloylated derivatives of tormentic.

According to an embodiment, step b) and step a) may be performed simultaneously, and the nutrient solution may be then substituted by the stimulating solution.

According to a preferred embodiment, the method for the preparation of a root extract of the plant Sanguisorba officinalis of the present invention comprises:
a) cultivating Sanguisorba officinalis under soilless conditions, in particular aeroponically,
b) stimulating the root of said plant,
c) solid/liquid extraction by maceration of the root obtained in step b)
d) recovering the extract obtained in step c), and
e) optionally, diluting or clarifying the extract recovered in step d) by successive filtrations.

The plants cultivated and stimulated are then typically subjected to a step c) of solid/liquid extraction by root maceration under given conditions in terms of solvent, of temperature and of extraction time, so as to obtain a root extract enriched in feruloylated derivatives of tormentic acid, and certainly with other secondary metabolites. Solid/liquid extraction is a solvent-based extraction known technique. The secondary metabolites released by the roots are recovered in the extraction solvent.

According to a preferred embodiment, the step c) of solid/liquid extraction of the roots stimulated during step b) may be preceded by an additional washing step in which the solvent diffused to the plants is clear water. The supply in the extraction solvent of the elements contained in the nutrient solution or in the stimulating nutrient solution is thus limited during the step of soaking in the solvent.

According to an embodiment, the root maceration may be performed on cut and then dried and optionally milled roots. The drying method can be any suitable typical known drying method, and notably by placing the roots biomass at a temperature of between 30° C. and 60° C. for 24 hours to 72 hours, preferably in a dry environment.

Root biomass may notably be dried in a ventilated oven. The milling of the root biomass may be performed by implementing any typical milling known method, and notably by placing the root biomass in a ball mill or a knife mill or a hammer mill. The plants, after the step of cutting the roots, are returned to aeroponic culturing according to steps a) and optionaly b) so as to recommence their root growth and to promote the production by the roots of secondary metabolites.

According to a preferred embodiment, the method may comprise a step of cutting the roots and drying the cut roots, prior to the step of maceration, the maceration being performed by placing the cut dried roots in contact with a solvent.

According to a preferred embodiment, the step c) of solid/liquid extraction may comprise placing the roots in contact with a solvent selected from water, alcohols, glycols, or a mixture thereof, and dicaprylyl ether, preferably bio-based dicaprylyl ether. Accordingly, alcohol may be preferably selected from ethanol and methanol, used pure or in the form of an aqueous solution, this comprising 10% to 99,9%, more preferably between 40% and 90%, and particularly between 50% and 85% of alcohol. Glycol may be selected from dipropylene glycol, propane-1,3-diol, propane-1,2-diol, pentylene glycol, 1,3-butanediol and glycerol, and may be used pure or in the form of an aqueous glycol solution, this comprising 10% to 99,9%, preferably between 70% and 99,9% of glycol.

Preferably, solvents may be glycols, which may be, for example, 1,3-propanediol, 1,3-butanediol or glycerol. In a preferred embodiment, the solvent may be selected from 1,3-propanediol. Particularly preferably, the solvent is bio-based 1,3-propanediol.

According to an embodiment, the step c) may comprise placing in the roots in contact, for example by maceration, with the solvent for a time between 30 min and 48 hours, preferably between 1 hour and 24 hours. In such case, the operating temperatures may be between 20° C. (room temperature) and 80° C., preferably between 40° C. and 60° C.

According to a particular embodiment, for the root maceration of dried roots, the ratio of the amount of dried roots and the amount of solvent may be range between 1 kg of dried roots/10 kg of solvent to 1 kg of dried roots/100 kg of solvent, more preferably between 1 kg of dried roots/20 kg of solvent and 1 kg of dried roots/40 kg of solvent.

By “root maceration in solvent”, as for an aspect of the invention, is meant a root maceration for which the solvent placed in contact with the roots is a solvent as which can be, for example, ethanol used pure or in the form of an aqueous solution, this comprising between 50% and 85% of ethanol, dicaprylyl ether used pure, 1,3-butanediol used pure or in the form of an aqueous solution, this comprising between 50% and 85% of 1,3-butanediol, 1,3-propanediol used pure or in the form of an aqueous solution, this comprising between 50% and 85% of 1,3-propanediol. More preferably the solvent is a bio-based 1,3-propanediol used pure or in the form of an aqueous solution, this comprising between 50% and 85% of bio-based 1,3-propanediol.

According to an embodiment, the method may comprise one or more additional steps of treating said plant root extract, which may be selected from the following known methods:
dilution, concentration,
one or more filtrations, in particular clarifying filtration and/or sterilizing filtration,
solid/liquid extraction,
purification,
bleaching of the liquid root extract.

The present invention further refers to a root extract of Sanguisorba officinalis obtained by the method as detailed above.

The present invention further refers to a cosmetic or a dermatological composition comprising:
a root extract of Sanguisorba officinalis according to the above aspect of the invention, or root extract of Sanguisorba officinalis obtained from a method defined above; and
at least a cosmetically or dermatologically acceptable ingredient other than the extract of Sanguisorba officinalis,

wherein the composition is a composition for topical use selected from the group consisting of a solution, a suspension, an emulsion, a cream, a paste, a gel, a lotion, a powder, a soap, a surfactant-containing water, an oil, a shampooing, and a spray, or wherein the composition is a nutraceutical composition which is administered orally.

According to a preferred embodiment of the composition, said root extract of Sanguisorba officinalis may represent 0.0001 to 15%, preferably 0.001 to 10%, more preferably 0.01 to 5% by weight, relative to the total weight of the cosmetic or dermatological or nutraceutical composition.

The modes of administration, the dosages and the optimal dosage forms of the cosmetic compositions according to the invention can be determined according to the criteria generally taken into account in the establishment of a cosmetic treatment adapted to a subject such as, for example, the type of skin. Depending on the type of administration desired, the cosmetic composition according to the invention may further comprise at least one cosmetically acceptable excipient. The cosmetic composition according to the present invention may further comprise at least one adjuvant cosmetically known in the art, chosen from thickeners, preservatives, perfumes, dyes, chemical or mineral filters, moisturizing agents, thermal waters, etc.

Accordingly, the cosmetic composition of the invention may further comprise other cosmetically active agents other than a root extract of Sanguisorba officinalis as defined above, such as other anti-aging agents, or moisturizing agents, agents having calming, soothing or relaxing activity, agents that stimulate cutaneous microcirculation, sebo-regulating agents for the care of oily skin, cleaning or purifying agents, anti-radical agents, anti-inflammatory agents, chemical or mineral sunscreens, etc.

Suitable cosmetic excipients are those known in the art. For example, suitable cosmetic excipients may be chosen from polymers, silicone compounds, surfactants, rheology agents, humectants, penetration agents, oily components, waxes, emulsifiers, film-forming agents and perfumes, electrolytes, pH adjusters, antioxidants, preservatives, dyes, pearlescent agents, pigments and mixtures thereof.

Accordingly, the cosmetic composition of the invention is advantageously intended for topical application. It may in particular be in the form a cream, a milk, a lotion, a gel, a serum, a spray, a mousse, a solution, an ointment, an emulsion, a patch or a mask.

Suitable dermatological excipients are also those known in the art. For example, suitable dermatological excipients may be identical as those intended for use in cosmetics.

Suitable nutraceutical excipients are known in the art. Examples may include water soluble polymers like cellulosic polymers, acrylate polymers and copolymers, polyvinylpyrrolidones, water soluble polyethylene glycols, vinyl copolymers. The nutraceutical composition may be for example a food supplement in the form of a solid, coated or non-coated tablet, liquid, powder, soft or hard gelatin capsule, etc.

It will be understood that the definitions and preferred embodiments made in the context of the root extract of Sanguisorba officinalis according to an aspect of the invention mutatis mutandis apply to a composition comprising said root extract.

The present invention also refers to the (cosmetic) use root extract of Sanguisorba officinalis according to an aspect of the invention as defined above as an active ingredient for skin care and/or scalp care, particularly for promoting the light-related human being mood by stimulating the production of at least one well-being hormone in the skin cells.

Advantagesously, the root extract of Sanguisorba officinalis according to an aspect of the invention as defined above is appropriately useful in the context of well-ageing or healthy ageing which are new concept of lifestyle as the said extract is able to stimulate well-being hormones, to prevent deleterious effects due to exogenous (e.g. UV-rays) and endogenuous factors on skin, and hence influencing the global skin homeostasis and impaciting both health and beauty.

Accordingly, the root extract of Sanguisorba officinalis of the present invention may be capable to reproduce or to mimic the beneficial effects of the light, preferably sunlight on the body, preferably on the skin, even in absence and/or in weak light exposure conditions. These light-related beneficial effects are obtained, even in absence and/or in weak light exposure conditions. Indeed, in light exposition conditions, the beneficial effects are much more better.

According to an embodiement, the use may be for promoting the production of at least one well-being hormone in the skin cells, for promoting or stimulating the expression of the light transducting molecules, preferably peropsins (RRH), in the skin cells, for maintaining or restoring the circadian rhythm of clocks proteins in skin cells, for stimulating the vitamin D downstream biological pathways, or a combination thereof.

Accordingly, the use for skin care may be additionally for preventing or delaying the signs of photoaging on the skin, for preventing or treating the UV-related skin damages, or a combination thereof.

According to an embodiment, the use for skin care may be for decreasing and/or preventing carbonylation of proteins in skin cells.

According to an embodiment, preventing or delaying the signs of photoaging on the skin may be for improving the mechanical properties of the skin, particularly for improving the skin elasticity, for improving the complexion of the skin, for increasing the dermis density, for maintaining or restoring the integrity of the skin cells, for maintaining or restoring the skin barrier function, for promoting the hydration of the skin, for improving the skin tone, for preventing the formation of fine lines and wrinkles, or a combination of two or more thereof.

As used herein, the term “well-being hormone” or “good mood hormone” or “happiness hormone” may be understood interchangeably in the broadest sense as generally understood in the art as a molecule (e.g., hormone consisting of proteins or peptides, or may be a chemical molecule) enabling to provide a well-being or a soothing effect in a subject.

By “weak light exposure conditions”, in the overall context of the present invention, is meant that the luminosity received by the body, by the skin or the scalp may be more than 5 lux, preferably more than 200 lux, even more preferably more than 5000 lux. In contrary, for example during a light-therapy, the intensity of the light may be roughly more than about 5000 lux, preferably more than about 10000 lux.

Advantageously, the light may be any light which may be of natural origin such as sunlight, daylight or artificial light emitted by any light source, for example emitted by a lamp or like. Preferably, the light is sunlight or daylight.

By “light related beneficial effects”, in the overall context of the present invention, is meant, for example, well-being promotion, mood control, and relaxation feelings. These effects may be obtainable, for example, by measuring the serotonin and melatonin in the plasma, as well as the expression level of peropsin (RRH) in the skin cells. It may also include the circadian rhythm regulation, the brain activity promotion, and the sleep modulation. Also, it is meant increasing the activity of vitamin D the skin cells for supplying the body's requirement.

By “downstream biological pathway”, in the overall context of the present invention, is meant the transport of the active forms of vitamin D by its nuclear receptor (VDR: vitamin D-receptor) and its carrier (DBP: vitamin D-binding protein) to exert its biological effects on the body. Hence, vitamin D may be distributed from the skin and/or the scalp to the all-body organs thanks to DBP.

It will be understood that the definitions and preferred embodiments made in the context of the use of the root extract of Sanguisorba officinalis according to the above aspect of the invention mutatis mutandis apply to the use of the said root extract of Sanguisorba officinalis. Particularly, the term “active ingredient”, in the overall context of the present invention, may be understood in the broadest sense as a component that may exhibit the desired and intended aforementioned activities, alone or may exhibit the activity together with one or more carriers that are themselves inactive.

The present invention also refers to a non-therapeutic method for preventing or delaying the appearance of skin aging effects and/or for promoting the well-being and/or relaxing feelings, said method comprises applying on at least a part of the skin and/or a scalp a root extract of Sanguisorba officinalis according to the above aspect of the invention.

### EXAMPLES

In the following examples, the root extract of Sanguisorba officinalis according to an aspect of the invention where the plant was cultivated under soilless conditions, in particular aeroponically will be called SORE.

SORE was prepared from plants whose seeds have been purchased from United Kingdom provider (Seedaholic) from 2015 to 2018. SORE was prepared from plants whose seeds have been purchased from a French provider (“Les Semences du Puy”) from 2019 to 2022. From 2019, the Sanguisorba officinalis plants were preserved and multiplied by the applicant.

### Example 1: Preparation of SORE and its Characterization

SORE was obtained according to the following process:
a) aeroponic cultivation of Sanguisorba officinalis with a nutrient medium of N/P/K composition corresponding respectively to 15/10/30 and to an electrical conductivity of between 0.4 and 1.6 mS/cm for a time of between 2 weeks and 6 weeks,
b) stimulation of the plant during 1 to 3 weeks with a nitrogen stress by using a nutrient solution of N/P/K composition comprising: less than 6% nitrogen, 15% phosphorus and 40% potassium, and with an electrical conductivity of 0.4 to 1.2 mS/cm,
   b′) rinsing with clear water followed by draining of the roots stimulated during step b)
   b″) cutting and drying the roots at a temperature between 30° C. to 60° C. for 24 to 72 hours in a ventilated oven
c) solid/liquid extraction by maceration of the dried roots in a solution of pure 1,3-propanediol during 2 to 24 hours, at a temperature of 50° C.
d) recovering of the root extract obtained during step c),
e) clarifying by filtration.

For the following, the feruloylated derivatives of tormentic acid having general formula I and formula II will be designated as FDTA-I and FDTA-II respectively. The tormentic acid will be designated as TA.

The quantification of the amount of TA and FDTA-I and FDTA-II in SORE was performed according to the protocol described in example 2.
dry extract content: 9.9 g/kg to 13.4 g/Kg
TA content: 119.9 mg/Kg to 217.2 mg/Kg (i.e. 1.21% to 1.67% of the dry extract)
FDTA-I content: 30.21 mg/Kg to 45.42 mg/Kg (i.e. 0.3% to 0.35% of dry extract)
FDTA-II content: 7.32 mg/KgL to 11.90 mg/Kg (i.e. 0.07% 0.09% of dry extract)

SORE obtained has 9 g to 13.4 g of dry extract per kilogram of extract and contained approximately 2% by the weight of total markers (FTA-I and FTA-II) relative to the total weight of the dry extract.

### Example 2: Method of Quantification of FDTA-I and FDTA-II

All samples were analyzed using the UHPLC Shimadzu Nexera X2 system (Shimadzu, Japan) with a PDA detector coupled to a mass spectrometer LCMS2020 (electrospray ionization in a negative ion mode, m/z 100-1000), using a Kinetex EVO C18 reverse phase column (150 mm×2.1 mm, 2.6 μm, Phenomenex, USA), maintained at 40° C. during all analyses. The mobile phase was composed of ultrapure water (Mili-Q, Merck Millipore)+0.1% of formic acid (Carlo Erba, France) (phase A) and pure acetonitrile (Sigma-Aldrich Chemie, Germany) (phase B), delivered at 0.5 ml/min with the gradient of the phase B as follows: 5-95% (0-10 min); 95% (10-13.5 min); 95-5% (13.5-13.55 min), 5% (13.55-15.1 min).

The quantification of FDTA-I and FDTA-II in the different extracts of Sanguisorba officinalis was carried out using a ferulic acid standard, which was prepared at the concentration of 100 mg/L in an ethanol/water mixture (70/30, v/v). The content in FDTA-I and FDTA-II was expressed in equivalent of ferulic acid in each extract sample.

In the context of the present disclosure, the concentration of FDTA-I and FDTA-II in the extracts of Sanguisorba officinalis expressed in mmol/L, was determined by measuring the areas of the peaks corresponding to FDTA-I and FDTA-II on HPLC chromatogram (at 330 nm) of said extract. FDTA-I and FDTA-II peak areas were then divided by the peak area of the standard solution comprising 0.515 mmol/L of ferulic acid and multiplied by molar concentration of ferulic acid standard (0.515 mmol/L). The values of molar concentration of FDTA-I and FDTA-II (expressed in mol/L) were converted to the mass concentrations (expressed in g/L) by multiplying them by the molecular weights of both compounds.

The quantification of FDTA-I and FDTA-II was conducted according to the following equation:

$$\frac{{Conc}_{FDTA}^{X} [ \frac{mol}{L} ] = ( {Conc}_{FA}^{STD} [ \frac{mol}{L} ] ) \cdot \frac{{A}_{FDTA}^{X} at 330 nm}{{A}_{FA}^{STD} at 330 nm}}{{Conc}_{FTA}^{X} [ \frac{g}{L} ] = {Conc}_{FTA}^{X} [ \frac{mol}{L} ] \cdot {MW}_{FDTA} [ \frac{g}{mol} ]}$$

where:

$${Conc}_{FDTA}^{X}$$

—concentration FDTA-I and FDTA-II (FDTAs) in the sample X;

$${Conc}_{FA}^{STD}$$

—concentration of the standard solution at 0.515 mmol/L;

$${A}_{FDTA}^{X}$$

—peak area of the compound FDTA in the sample X at 330 nm;

$${A}_{FA}^{STD}$$

—peak area of ferulic acid standard at 330 nm; corresponding to the concentration of the standard solution at 0.515 mmol/L;
M_{WFDTA}—molecular weight of FDTA

Tormentic acid quantification in the different extracts of Sanguisorba officinalis was carried out using TA standard prepared at the concentration of 100 mg/L in pure ethanol. In the context of the present disclosure, the concentration of TA in the extracts of Sanguisorba officinalis was determined by measuring the peak area corresponding to the TA on the HPLC chromatogram (190 nm) of said extract. The areas of TA peak in the extract samples were divided by the peak area of the standard solution of TA at 100 mg/L and multiplied by the mass concentration of the standard (100 mg/L), according to the following equation:

$${Conc}_{TA}^{X} [ \frac{g}{L} ] = ( {Conc}_{TA}^{STD} [ \frac{g}{L} ] ) \cdot \frac{{A}_{TA}^{X} at 190 nm}{{A}_{TA}^{STD} at 190 nm}$$

where:

$${Conc}_{TA}^{X}$$

—concentration of tormentic acid (TA) in the sample X

$${Conc}_{TA}^{STD}$$

—concentration of the standard solution of TA (100 mg/L)

$${A}_{TA}^{X}$$

—peak area of TA in the sample X at 190 nm

$${A}_{TA}^{STD}$$

—peak area of TA standard at 190 nm, corresponding to the concentration of 100 mg/L;

The method of quantification of TA and FDTA-I and FDTA-II described above was used for all the following examples.

### Example 3: Phytochemical Analyses of FDTA-I and FDTA-II Amount in Different Extracts of Sanguisorba officinalis with Different Mode of Cultivation and in Different Tissues

Sanguisorba officinalis plants were cultivated aeroponically for 4 weeks with a 15/10/30 (N/P/K) culture medium and with an electroconductivity between 1,0 and 1.2 mS/cm, then with a defined solution nutrient, with an N/P/K composition corresponding to 0/15/40 and an electrical conductivity of between 1 and 1.2 mS/cm for a 2-week step of stimulating the roots. The roots that were harvested before changing the culture medium corresponded to the non-stimulated roots. Furthermore, available commercial dry roots of Sanguisorba officinalis, almost exclusively constituted by rhizomes, have been purchased (Xuewen Tang Bozhou Swanf Commerce & Trade) for evaluating their phytochemical composition.

Roots and aerial parts were cut, harvested, and dried for 48 hours at 50° C. in a ventilated oven and ground. For the preparation of each extract, 25 mg of ground roots in powder form or 25 mg of ground aerial parts in powder were macerated in 0.5 mL in an ethanol/water (70/30 v/v) with stirring at ambient temperature for 1 h. The samples were centrifuged and analyzed by HPLC-UV-MS.

The results are summarized in the Table 1 below:

TABLE 1
Concentrations of TA and FDTA-I and FDTA-II in different
extracts of root or aerial parts of Sanguisorba officinalis
cultivated in different conditions (n = 3).

| | TA | FDTA-I | FDTA-II |
|---|---|---|---|
| | (mg/L) | (mg/L) | (mg/L) |
| (A) Roots in aeroponic | 1012.5 ± (14.4) | 18.7 ± (0.3) | 1.2 ± (0.02) |
| (non-stimulated) | | | |
| (B) Roots in aeroponic | 712.1 ± (10.7) | 40.7 ± (0.3) | 12.8 ± (0.1) |
| (stimulated) | | | |
| (C) Commercial roots | 7.7 ± (2.0) | 0.0 | 0.0 |
| (D) Aerial parts in | 48.9 ± (5.6) | 0.0 | 0.0 |
| aeroponic | | | |
| (non-stimulated) | | | |

It was observed that the extract of the roots of Sanguisorba officinalis cultured aeroponically contain both FDTA-I and FDTA-II.

Moreover, the concentration of FDTA-I and FDTA-II was strongly increased by a step of stimulation:
by a factor of 2.17 for FDTA-I between non-stimulated and stimulated roots of plants cultivated in aeroponic conditions,
by a factor of 10.66 for FDTA-II between non-stimulated and stimulated roots of plants cultivated in aeroponic conditions.

Interestingly, it was observed a decrease in TA amount in stimulated roots compared to non-stimulated roots. This suggest that TA may has been conjugated with ferulic acid upon stimulation thus increasing FDTA-I and FDTA-II.

This study also confirmed that:
aerial parts from Sanguisorba officinalis do not contain FDTA-I and FDTA-II whether they are cultivated in soil or in aeroponic conditions,
FDTA-I and FDTA-II were not detected, according to the protocol study performed, in dry roots commercially available (exclusively rhizomes).

These results highlighted one of the advantages of cultivating Sanguisorba officinalis roots in aeroponic conditions.

### Example 4: Extraction Process of TA and FDTA-I and FDTA-II from SORE

Extracts were prepared from roots of Sanguisorba officinalis cultivated aeroponically with a stimulation step according to the example 3

### A) Comparison of Different Extraction Solvents

The different solvents chosen for the solid/liquid extraction by maceration according to step c) of the method for preparing a root extract are an ethanol/water at 70/30 (v/v), pure water, a mixture of propane-1,3-diol/water at 30/70 (v/v) or at 50/50 (v/v) and at 70/30 (v/v), pure propane-1,3-diol, a mixture of 1,3-butanediol/water at 80/20 (v/v) and a pure dicaprylyl ether.

The extraction rates of TA and FDTA-I and FDTA-II were compared to those of the ethanolic extraction carried out under the same conditions explained in example 3 except for the maceration duration (4 h in this example). The content of TA, FDTA-I and FDTA-II in each sample was measured according to the protocol described in example 2. Table 2 below describes the results:

TABLE 2
Results of TA and FDTA-I and FDTA-II extraction yields with different solvents,
the extraction yields are calculated relative to ethanol (70%).

| | Amount of: (%) | | | | | | | |
|---|---|---|---|---|---|---|---|---|
| | Solvent | | | | | | | |
| | Ethanol | Water | 1,3-PD | 1,3-PD | 1,3-PD | 1,3-PD | 1,3-BD | DE |
| | (70%) | (100%) | (30%) | (50%) | (70%) | (100%) | (80%) | (100%) |
| FDTA-I | 100 | 0 | 0 | 0 | 19 | 64 | 57 | 16 |
| FDTA-II | 100 | 0 | 0 | 0 | 0 | 75 | 59 | 24 |
| TA | 100 | 13 | 3 | 5 | 12 | 53 | 50 | 5 |
| Total FDTA-I and | 100 | 13 | 3 | 5 | 12 | 57 | 52 | 9 |
| FDTA-II | | | | | | | | |

(PD: propanediol; BD: butanediol; DE: dicaprylyl ether)

Ethanol/water mixture at the ratio of 70/30 can be considered as a reference solvent for a skilled person. Results shown in table 2 demonstrated that among all solvents tested, pure propane-1,3-diol have the best extraction rates of TA and FDTA-I and FDTA-II. Pure propane-1,3-diol was chosen for the following examples.

### B) Comparison of Different Extraction Methods Independent of the Solvent

In order to improve the extraction of TA and FDTA-I and FDTA-II in SORE, it was performed a comparison of different extraction methods with pure propane-1,3-diol as maceration solvent. Extractions were carried out under the same conditions than explained in example 3 except for the extraction temperature (25° C. and 50° C.) and the extraction lasted 2H, 4H and 24H

The content of TA and FDTA-I and FDTA-II in each sample was measured according to the protocol described in example 2. The extraction yields were calculated relative to 50° C. or relative to 24 h of extraction.

For the extraction temperature experiments, the extract used for the reference (100) had the following characteristics:
dry extract content: 7.06 g/Kg
TA content: 105.85 mg/Kg (i.e. 1.50% of the dry extract)
FDTA-I content: 40.10 mg/Kg (i.e. 0.57% of dry extract)
FDTA-II content: 29.27 mg/Kg (i.e. 0.41% of dry extract)

For the extraction duration experiments, the extract used for the reference (100) had the following characteristics:
dry extract content: 13 g/Kg
TA content: 217.20 mg/Kg (i.e. 1.67% of the dry extract)
FDTA-I content: 45.42 mg/Kg (i.e. 0.35% of dry extract)
FDTA-II content: 10.64 mg/Kg (i.e. 0.08% of dry extract)

Tables 3 and 4 below describe the results:

TABLE 3
Results of TA and FDTA-I and FDTA-II extraction yields with two
different extraction temperatures. The extraction lasted 2 H.

Extraction temperature

| | 25° C. | 50° C. |
|---|---|---|
| Amount of FDTA-I (%) | 33.8 | 100 |
| Amount of FDTA-II (%) | 22.3 | 100 |
| Amount of TA (%) | 80.0 | 100 |
| Total amount of FDTA-I and FDTA-II | 59.8 | 100 |

These results confirmed that the optimal temperature to extract TA and FDTA-I and FDTA-II is 50° C. It enables the following gain compared to 25° C.:
increased yield of FDTA-I: multiplied by 3.
increased yield of FDTA-II: multiplied by 4.5.
increased yield of TA: multiplied by 1.25.
increased yield of FDTA-I and FDTA-II: multiplied by 1.7

TABLE 4
Results of TA and FDTA-I and FDTA-II extraction yields with different
extraction duration. The extraction temperature was 50° C.

Extraction duration

| | 2 h | 4 h | 24 h |
|---|---|---|---|
| Amount of FDTA-I (%) | 66.5 | 79.5 | 100 |
| Amount of FDTA-II (%) | 68.8 | 75.9 | 100 |
| Amount of TA (%) | 55.2 | 75 | 100 |
| Total FDTA-I and FDTA-II (%) | 57.6 | 76 | 100 |

These results confirmed that the optimal duration to extract TA and FDTA-I and FDTA-II was 24 h. It enables the following gain compared to 2 h and 4h:
increased yield of FDTA-I: multiplied by 1.5 and 1.25 respectively.
increased yield of FDTA-II: multiplied by 1.45 and 1.3 respectively.
increased yield of TA: multiplied by 1.8 and 1.3 respectively.
increased yield of TA and FDTA-I and FDTA-II: multiplied by 1.8 and 1.3 respectively.

### Example 5: SORE Provides Well being and Relaxing Feeling Through the Upregulation of the Expression of Serotonin and Melatonin

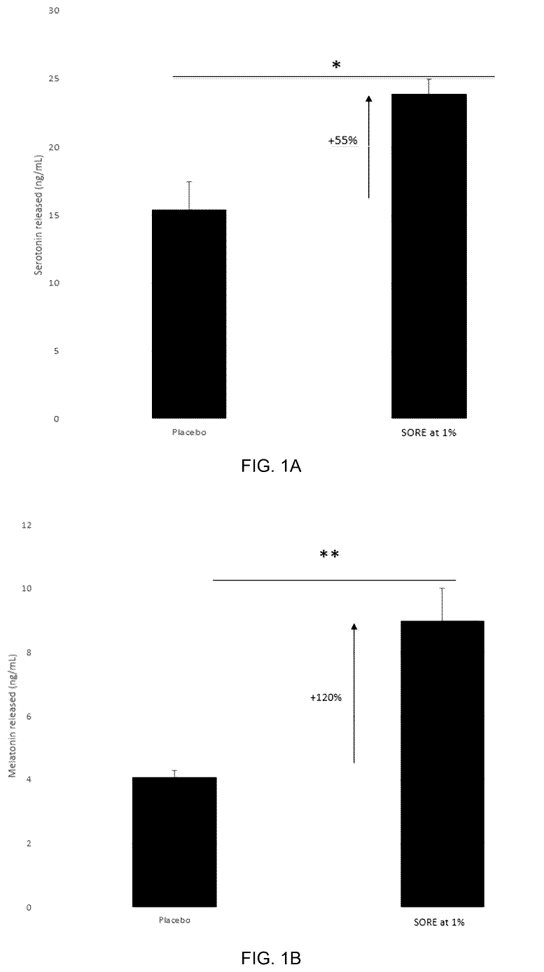

Serotonin also called “the good-mood hormone” or “well being hormone” is the body's natural mood booster has been shown to be upregulated by sunlight. The skin may play a central place in the link between light and mood, in which serotonin could be of great importance. Thus, experiments showed that SORE was able to mimic light transduction signal in the skin. It was wondering if SORE topically applied on skin may be capable to upregulate serotonin expression in absence of sunlight exposure. To do so, serotonin was assessed in culture media of skin explants treated 3 days with SORE at 1% in absence of light using a commercial ELISA kit (Biovison, Abcam) according to the manufacturer's instructions. Interestingly, SORE was shown to significantly stimulate the production of serotonin by skin explants by +55% (FIG. 1A).

For transcriptomic analysis, after 24 hours of tissue culture, total RNAs were extracted using RNeasy Mini kit from Qiagen. Their concentrations and integrities were analysed by spectrophotometry and capillary electrophoresis. Transcriptomic analysis was performed on Affymetrix human Clariom S arrays according to the Affymetrix user manual. To analyse data, all statistically significantly regulated genes were analysed using the DAVID bioinformatics resources. The tool identifies functional regulated pathways from large genes or proteins datasets.

The transcriptomic data (Table 5) showed that SORE may upregulate the mRNA expression of 5-hydroxytryptamine receptor 3A, one of the several receptors for 5-hydroxytryptamine (serotonin).

TABLE 5
upregulation of mRNA expression of HTR3A

| Gene | Fold change | p-value |
|---|---|---|
| HTR3A (5-hydroxytryptamine receptor 3A) | 1.57 | 0.021 |

Serotonin is the precursor of melatonin, and melatonin is linked to the quality of sleep, itself being linked to well being. Melatonin secretion is dependant of the light/dark cycle. As melatonin also stimulates antioxidant and DNA repair systems and as its precursor, serotonin was upregulated by SORE, melatonin was assessed in culture media of skin explants treated 5 days with SORE in absence of light using a commercial ELISA kit (Abbexa) according to the instructions of manufacturer. Interestingly, SORE was shown to significantly stimulate the production of melatonin by skin explants at day 5 by +120% (FIG. 1B).

The well being effect of SORE was then demonstrated at the clinical level on two clinical studies.

The gel cream containing SORE at 1% (cf. table 9) was used in a double blind and vehicle controlled clinical study. In this regard, the recommendations of the Declaration of Helsinki and the guidelines of the International Conference on Harmonization Good Clinical Practice were observed as applicable to a non-drug study.
In the first study (proof of concept study): 10 caucasian volunteers, aged 32 to 58 years old, composed of 4 women and 6 men applied in entire face, twice a day for 5 days the SORE containing gel cream. In this study the same volunteers tested the SORE containing gel cream and the placebo. There was a wash out period of 5 days between the two sessions of the evaluation of the SORE containing gel cream. The order of use of SORE containing gel cream and placebo were randomly assigned to volunteers.
In the second study: 36 caucasian women, aged 30 to 50 years old. The volunteers enrolled in this study declared to be depressed. Volunteers presented a poor face skin complexion, and a lack of skin brightness. The panelists were randomized in two groups: placebo formula (21 volunteers) and SORE formulated (15 volunteers) and were asked to use SORE containing gel cream or the placebo on the entire face, twice a day for 28 days.

To study the improvement of the mood (well being) of the volunteers in these two studies, it was used a headset device from the company MyBrain Technologies able to measure electric signals emitted by brain during its activity. These brain electric signals are measured at the surface of the head thanks to the captors of the headset. A proprietary algorhythm calculates a relaxation index (the higher the value, the more relaxed the volunteer). The measurements were done at least 1 hour after the application of the product to avoid the influence of sensitive characteristics of the formula (perfume, texture, . . . ). Thus, the effect observed is due to SORE itself.

For the study 1, the measurements were done on volunteers at day 0 (before the use of the SORE containing gel cream) then at day 1 and 5 after twice daily application of SORE containing gel cream.

For the study 2, the measurements were done at day 0 and after 7 days of twice a day application of SORE containing gel cream.

To normalize the values of the initial state of each volunteer, % variation was calculated for each volunteer regarding its initial state, according to:

(Final value—Day0value)/Day0value*100

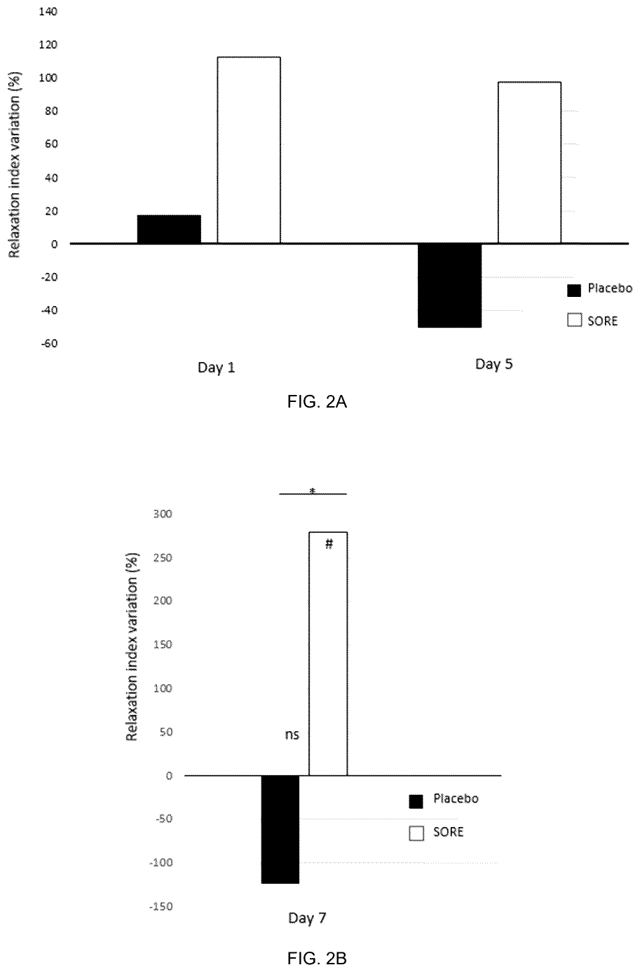

SORE formulated, used topically on face, improves the relaxation of the volunteers as soon as after 1 day of use, in contrast to placebo. Moreover, this effect is maintained after 5 days of use. FIG. 2A shows that SORE improved relaxation: +112% at day 1 and +97% at day 5, whereas placebo did not: +17% at day 1 and −50% at day 5.

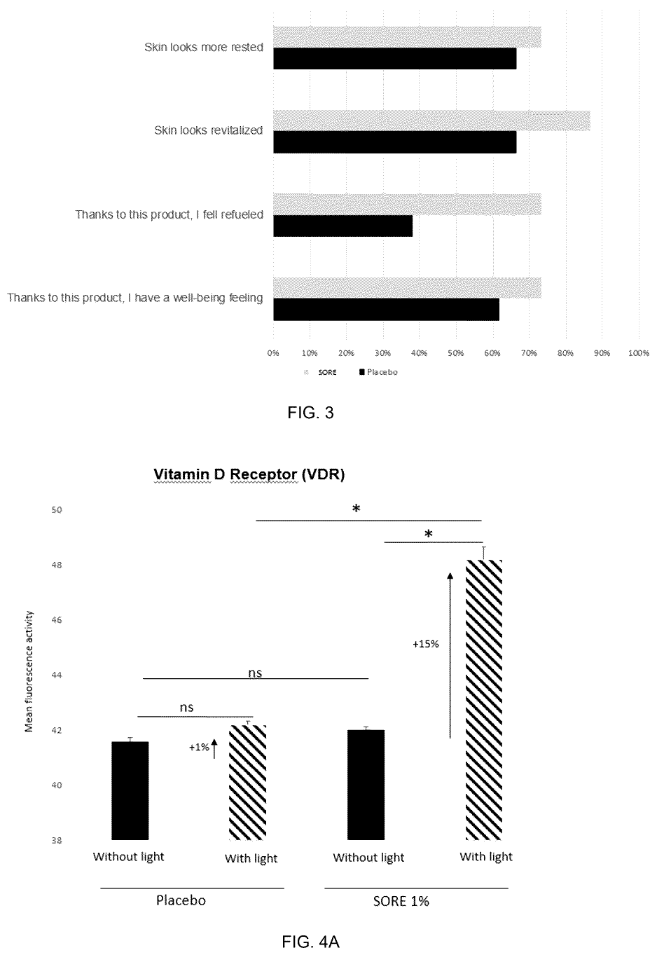

In the second study, it was confirmed the well being effect of SORE formulated observed in the first clinical study. Indeed, the relaxation parameter was improved after 7 days of SORE formulated used in comparison to placebo control. FIG. 2B shows that SORE formulated was able to significantly improve relaxation (+279% #p<0.07) at day 7, whereas placebo did not (−123.6% ns). The difference of effect of SORE was significant relatively to the one of placebo (* p<0.05). It showed that the effects observed in vitro on serotonin secretion and well being was confirmed in vivo on relaxation. Moreover, the volunteers' self-evaluation confirmed the well being effect of SORE formulated (FIG. 3). The volunteers found themselves, refueled and expressed an improvement of well being sensation. Moreover, the volunteers found their skin more rested and revitalized, two signs altered in depressed situations.

### Example 6: SORE Upregulates Vitamin D Downstream Biological Pathways

Indeed, as vitamin D cannot be produced by the skin in absence of sunlight (especially UVB), it was wondering whether SORE may potentialize the effect of sunlight by upregulating vitamin D downstream biological pathways. Indeed, the active forms of vitamin D need VDR and the carrier DBP (vitamin D-binding protein DBP) to exert their biological effects. Vitamin D may be distributed from the skin to the all-body organs thanks to vitamin D-binding protein (DBP). Vitamin D may also have direct beneficial effects in skin via its nuclear receptor (VDR). Thus, the effects of SORE on vitamin D downstream biological pathways (VDR and DBP) were assessed both at the transcriptomic level in skin explants after 24 h of SORE treatment and at the proteomic level in skin explants after 5 days of SORE treatment coupled or not with light-therapy exposure.

The related effect of SORE formulated at 1% (cf. table 9) on vitamin D-binding protein (DBP) was studied in transcriptomic analysis, as described in example 5, and light-therapy coupled effect was studied for Vitamin D receptor (VDR).

TABLE 6
skin explants treated by SORE for 24 hours expressed
significant stimulation of DBP mRNA expression

| Gene | Fold change | p-value |
|---|---|---|
| GC vitamin D binding protein (DBP) | 1.72 | 0.004 |

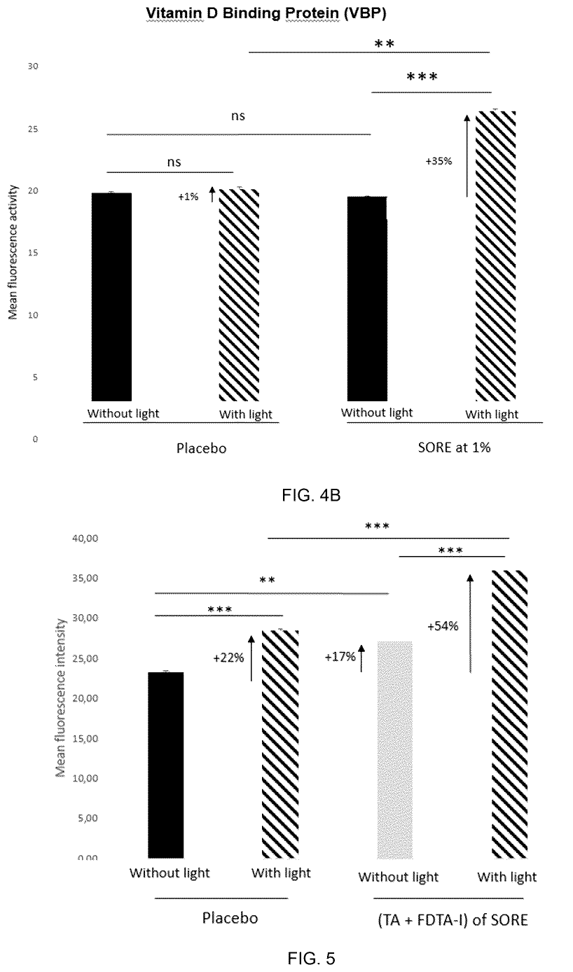

FIG. 4 shows in skin explants simultaneously treated with light (without UV-B rays) and SORE formulated at 1% (cf. table 9) for 5 days an upregulation of the protein's expression of VDR (+15%: FIG. 4A) and DBP (+35%: FIG. 4B). All these results confirmed that SORE may improve the distribution of the vitamin D produced in skin to other tissues through the upregulation of its carrier (DBP). Moreover, experiments demontrated that SORE may also improve the biological effects of vitamin D in the skin as it was shown an upregulation of VDR.

### Example 7: Mixture of FDTA-I and TA from SORE Mimics Light Effects on the Skin

SORE was formulated at 0.75% by weight in a gel cream (Table 7). The biological effects of SORE compared to placebo were assessed ex vivo on human skin explants from a female Caucasian donor aged 39 years old by transcriptomic analysis. Briefly, formulas were topically applied at 2 mg/cm² on skin explants. Explants were cultured for 24 h at 37° C. with 5% CO₂.

TABLE 7
Gel cream formulation containing 0.75% by weight of SORE

| INCI | Placebo (%) | SORE (%) |
|---|---|---|
| Water | 96.000 | 95.250 |
| Ammonium | 0.800 | 0.800 |
| Acryloyldimethyltaurate/ | | |
| VP Crosspolymer | | |
| Dicaprylyl Ether | 1.000 | 1.000 |
| Citric Acid (and) Sodium Citrate | 1.000 | 1.000 |
| SORE | 0.000 | 0.750 |
| Phenoxyethanol (and) | 1.100 | 1.100 |
| Methylparaben (and) | | |
| Ethylparaben | | |
| Fragrance | 0.100 | 0.100 |

SORE similarly to visible light induced light transduction signal (but in absence of light). The transcriptomic study, as described in example 5, showed that SORE induced in skin the expression of photoreceptors known to be induced by visible light confirming that SORE may be able to trigger light signal transduction even in absence of light exposure. Indeed, visible light has been described to stimulate opsin photoreceptors as Perospin (RRH) or Rhodopsin to transduct light signal. The transcriptomic skin analysis showed an enrichment of transcripts involved in light transduction signal and in the homeostasis of photoreceptors usually found in the retina (Table 8):

TABLE 8
Stimulation of light perception related genes
in the skin induced by SORE formulated

| Gene | Fold change | p-value |
|---|---|---|
| RRH (Visual pigment-like | 1.63 | 0.022 |
| receptor peropsin) | | |
| OR56A5 (Olfactory receptor | 1.61 | 0.063 |
| family 56 subfamily A member 5) | | |
| SAG (S-antigen visual arrestin) | 1.66 | 0.004 |

RRH plays a role in retinal pigment epithelium physiology either by detecting light directly or by monitoring the concentration photoreceptor-derived compounds. It is also expressed in skin, where it may be related to phototransduction of short-wavelength, violet light, suggesting that the skin may harbour photoreceptors that may indeed sense and distinguish various light qualities.
OR56A5 is described as muted in retinitis pigmentosa which is a disease associated with loss vision. ORA5 is involved in signal transduction of UV light.
SAG binds to photoactivated, phosphorylated Rho and terminates Rho signaling via G-proteins by competing with G-proteins for the same binding site on Rho. It plays a role in preventing light-dependent degeneration of retinal photoreceptor cells.

Then, it was demontrated the ability of SORE to induce the light transduction signal without light exposition on skin explants similarly to visible light. In other words, SORE can induce the expected biological effects of the light on the skin without light.

An additional ex vivo experiments (skin from a female donor aged 41 years old) in presence or not of light by using a commercial light therapy device (Dayvia White072) were performed. This device is dedicated to exposition of people suffering from seasonal bad mood. Briefly, gel creams containing SORE at 1% or placebo (cf. table 9) were topically applied at 2 mg/cm² on skin explants. The explants were exposed to the light therapy device. The device delivered a light of 10000 Lux at a distance of 21 cm without UV nor infrared. 10000 Lux is the light intensity recommended for light therapy. Skin explants received a placebo, or SORE and then treated to the light of this device at a distance of 21 cm for 20 minutes. Non-exposed topically treated explants were also cultured for reference. Daily expositions were performed as well as daily topical applications of placebo or SORE. The topical applications were done just after light exposure. Explants were cultured for 5 days at 37° C. with 5% CO₂.

At the end of the experiment, explants were formalin fixed then embedded in paraffin. Thin sections were processed for RRH immunofluorence. Expression levels were estimated by fluorescence quantification with Image J. FIG. 5 shows as expected that light upregulated the expression of RRH. Moreover, SORE may stimulate the expression of RRH (+17%) in absence of light, showing its ability to mimic light effect on this photoreceptor and may initiate a light-like effect. This result confirmed the transcriptomic data. In addition, SORE may be also able to potentiate the light perception throught RRH (+54%), as its expression was higher in SORE treated light-exposed skin explants than in placebo treated light exposed ones.

TABLE 9
Gel cream containing SORE at 1% by weight and placebo

| INCI | Placebo (%) | SORE (%) |
|---|---|---|
| Water | 96.000 | 95.05 |
| Ammonium Acryloyldimethyltaurate/ | 0.800 | 0.80 |
| VP Crosspolymer | | |
| Dicaprylyl Ether | 1.000 | 1.00 |
| Citric Acid (and) Sodium Citrate | 1.000 | 1.00 |
| SORE | 0.000 | 1.00 |
| Phenoxyethanol (and) | 1.100 | 1.10 |
| Methylparaben (and) | | |
| Ethylparaben | | |
| Fragrance | 0.100 | 0.05 |

### Example 8: SORE Regulates the Skin Cells' Circadian Rhythm

The results were obtained from human skin explants experiments described in example 5.

TABLE 10
key circadian rhythm genes upregulated by
SORE formulated at 0.75% (cf. table 7).

| Genes | Fold change | p-value |
|---|---|---|
| TOP2A (DNA topoisomerase | 1.84 | 0.013 |
| II alpha) | | |
| SUV39H2 (suppressor of | 1.58 | 0.009 |
| variegation 3-9 homolog 2) | | |
| SFPQ (splicing factor proline | 1.38 | 0.030 |
| and glutamine rich) | | |
| PSPC1 (paraspeckle component 1) | 1.28 | 0.038 |
| TIMELESS (timeless | 1.77 | 0.017 |
| circadian regulator) | | |
| HNRNPU (heterogeneous nuclear | 1.27 | 0.024 |
| ribonucleoprotein U) | | |
| PASD1 (PAS domain | 1.40 | 0.035 |
| containing repressor 1) | | |

TOP2A plays a role in regulating the period length of ARNTL/BMAL1 transcriptional oscillation and influences the circadian period.
SUV39H2 may be recruited by the large PER complex to the E-box elements of the circadian target genes such as PER2 itself or PER1.
Both SFPQ, and PSPC1 may regulate the circadian clock by repressing the transcriptional activator activity of the CLOCK-ARNTL/BMAL1 heterodimer. They are required for the transcriptional repression of circadian target genes, such as PER1, mediated by the large PER complex through histone deacetylation.
TIMELESS is involved in the determination of period length and in the DNA damage-dependent phase advancing of the circadian clock. Timeless, negatively regulates CLOCK|NPAS2-ARTNL/BMAL1|ARTNL2/BMAL2-induced transactivation of PER1 possibly via translocation of PER1 into the nucleus.
HNRNPU has many functions, including participation in the circadian regulation of the core clock component ARNTL/BMAL1 transcription.
PASD1 functions as a suppressor of the biological clock that drives the daily circadian rhythms of cells throughout the body. It may act as a nuclear repressor of the CLOCK-ARNTL/BMAL1 heterodimer-mediated transcriptional activation of the core clock components.

All these results demonstrated that SORE may modulate the skin cell's circadian rhythm, which is fundamental in regulating a wide range of cellular, metabolic, physiological, and behavioral activities in mammals.

### Example 9: SORE Preserves Cellular Integrity by the Induction of DNA Repair Mechanisms

The results were obtained from human skin explants experiments described in example 5. SORE has shown cellular protective effect by upregulating DNA repair players (Table 11).

TABLE 11
upregulation of DNA repair related genes on the skin explants
treated by a SORE formulated at 0.75% (cf. table 7).

| Genes | Fold change | p-value |
|---|---|---|
| MBD4 (methyl-CpG binding domain 4, DNA glycosylase) | 1.47 | 0.014 |
| TREX1 (three prime repair exonuclease 1) | 1.37 | 0.029 |
| BOD1L1 (biorientation of chromosomes in cell division 1 | 1.30 | 0.041 |
| like 1) | | |
| RAD51B (RAD51 paralog B) | 1.61 | 0.020 |
| RAD51AP1 (RAD51 associated protein 1) | 1.68 | 0.045 |
| KIN (Kin17 DNA and RNA binding protein) | 1.55 | 0.040 |
| MSH2 (mutS homolog 2) | 1.63 | 0.048 |
| FANCD2 (FA complementation group D2) | 1.97 | 0.002 |
| RPA3 (replication protein A3) | 1.69 | 0.033 |
| POLI (DNA polymerase iota) | 1.43 | 0.041 |
| SLF1 (SMC5-SMC6 complex localization factor 1) | 2.04 | 0.005 |
| XPA (XPA, DNA damage recognition and repair factor | 1.71 | 0.009 |
| NSMCE4A (NSE4 homolog A, SMC5-SMC6 complex | 1.69 | 0.045 |
| component) | | |
| ATR (ATR serine/threonine kinase) | 1.62 | 0.033 |
| PRIMPOL (DNA-directed primase/polymerase protein) | 1.69 | 0.033 |

MBD4 is a mismatch-specific DNA N-glycosylase involved in DNA repair. MDB4 has thymine glycosylase activity and is specific for G: T mismatches within methylated and unmethylated CpG sites. It also removes uracil or 5-fluorouracil in G: U mismatches.
TREX1 is a major cellular 3′-to-5′ DNA exonuclease which can digest single-stranded DNA (ssDNA) and double-stranded DNA (dsDNA) with mismatched 3′ termini.
BOD1L1 is a component of the fork protection machinery required to protect stalled/damaged replication forks from uncontrolled DNA2-dependent resection. It acts by stabilizing RAD51 at stalled replication forks and protecting RAD51 nucleofilaments from the antirecombinogenic activities.
RAD51B is involved in the homologous recombination repair (HRR) pathway of double-stranded DNA breaks arising during DNA replication or induced by DNA-damaging agents. It promotes the assembly of presynaptic RAD51 nucleoprotein filaments.
KIN is involved in DNA replication and the cellular response to DNA damage. It participates in DNA replication factories and create a bridge between DNA replication and repairs mediated by high molecular weight complexes.
MSH2 is a component of the post-replicative DNA mismatch repair system (MMR) which binds to DNA mismatches thereby initiating DNA repair.
FANCD2 is required for maintenance of chromosomal stability and is involved in the repair of DNA double-strand breaks, both by homologous recombination and single-strand annealing.
RAP3 is part of the heterotrimeric replication protein A complex (RPA/RP-A), that binds and stabilizes single-stranded DNA intermediates that form during DNA replication or upon DNA stress. Thereby, it plays an essential role both in DNA replication and the cellular response to DNA damage.
POLI is an error-prone DNA polymerase specifically involved in DNA repair.

Some upregulated systems of DNA repair are very specific of environmental damages and especially from UV light.
XPA is involved in DNA excision repair. It initiates repair by binding to damaged sites with various affinities, depending on the photoproduct and the transcriptional state of the region. Also, it is required for UV-induced CHEK1 phosphorylation and the recruitment of CEP164 to cyclobutane pyrimidine dimmers (CPD), sites of DNA damage after UV irradiation.
SLF1 plays a role in the DNA damage response (DDR) pathway by regulating postreplication repair of UV-damaged DNA and genomic stability maintenance. Compementarly, NSMCE4A, non-structural maintenance of chromosomes element 4 homolog A, is a component of the SMC5-SMC6 complex, which is involved in DNA double-strand breaks by homologous recombination.
ATR activates checkpoint signaling upon genotoxic stresses such as ionizing radiation (IR), ultraviolet light (UV), or DNA replication stalling, thereby acting as a DNA damage sensor.
PRIMPOL is a DNA primase and DNA polymerase required to tolerate replication-stalling lesions by bypassing them. It provides different translesion synthesis alternatives when DNA replication is stalled. It is able to synthesize DNA primers downstream of lesions, such as ultraviolet (UV) lesions, R-loops and G-quadruplexes, to allow DNA replication to continue.

Therefore, SORE is acting on several DNA repair components involved in regular or environmental-induced DNA damages. It may be a powerful protectant in conserving the DNA intergrity which may be compromised both by aging process and/or by environmental aggression as sunlight.

### Example 10: Anti-Collagenase Inhibiting Activity of FDTA-I and FDTA-II Purified from SORE

FDTA-I and FDTA-II were purified from stimulated roots of Sanguisorba officinalis, where the plant has been stimulated by using a nutrient medium without nitrogen (N/P/K 0/15/40) for 1 to 3 weeks. 10 g of dried and grinded (1 mm) roots were macerated in 100 ml of ethyl acetate for 24 h at 50° C. The purified fraction of FDTA-I and FDTA-II was obtained by preparative HPLC and characterized by mass spectrometry. Ferulic acid and TA have been purchased commercially. In addition, it was also included in the test ziyuglycoside I, an active compound isolated from Sanguisorba officinalis roots known to hold various activities including increased expression of type I collagen. For the comparison, SORE was obtained as presented in example 1. The anti-collagenase inhibiting activity was measured by following the kinetics of collagenase-driven degradation of collagenase substrate: FALGPA (N-[3-(2-Furyl) acryloyl]-Leu-Gly-Pro-Ala) into FAL (N-[3-(2-Furyl) acryloyl]-Leu) and Gly-Pro-Ala.

Briefly, a volume of 100 μL of 1.5 mM solution of FALGPA in 50 mM tricine buffer with 10 mM calcium chloride and 400 mM sodium chloride, pH 7.5 (Bachem, 4006713.0025) was mixed with a volume of 10 μL of test sample or pure solvent of the test sample (positive control), followed by a volume of 10 μL of 0.05 mg/ml of Clostridium histolyticum collagenase (type IA, Sigma-Aldrich, C9891). To determine the rate of enzymatic conversion, the content of FAL was quantified at the beginning of the reaction and 30 minutes after its initialization by stopping the enzymatic conversion of FALGPA by EDTA. To quench the enzymatic conversion, a volume of 50 μL of the test sample was mixed with a volume of 50 μL of EDTA (0.2 M in water). The content of FAL in the samples was then quantified by UHPLC method optimized to separate FALGPA and FAL. The analysis was conducted using Kinetex Biphenyl reverse phase column (150 mm×2.1 mm, 2.6 μm, Phenomenex, Torrance, USA), maintained at 40° C. The mobile phase was composed of water containing 0.1% vol. of formic acid (A) and pure acetonitrile (B), delivered at 0.5 ml/min with the gradient of B phase as follows: 5-41% (0-9 min); 41-90% (9-9.05 min); hold at 90% (9.05-11.50 min); 90-5% (11.50-11.55 min), hold at 5% (11.55-14.50 min). Sample injection volume was 5 μL and FALGPA/FAL detection at 338 nm. Residual activity and the inhibition degree of collagenase in the presence of test sample X was then calculated using the following equation:

$$ActR {\%}_{EchX} = \frac{{AUC}_{EchX , T = 30}^{FAL , 338 nm} - {AUC}_{EchX , T = 0}^{FAL , 338 nm}}{{AUC}_{BL , T = 30}^{FAL , 338 nm} - {AUC}_{BL , T = 0}^{FAL , 338 nm}} \cdot 100 \%$$

$$Inh {\%}_{EchX} = 100 \% - ActR {\%}_{EchX}$$

where:

ActR %—residual activity; Inh %—inhibition degree; AUC—peak area of FAL; EchX tested sample; BL—blank.

TABLE 12
Anti-collagenase inhibiting activity of FDTA-I and FDTA-
II, ziyuglycoside I, TA and ferulic acid at a concentration
of 25 μM and SORE at a concentration of 0.04%.

| | Residual activity, ActR (%) |
|---|---|
| SORE (0.04%) | 50 |
| Ziyuglycoside I (25 μM) | 106 |
| FDTA-I (25 μM) | 24 |
| FDTA-II (25 μM) | 64 |
| TA (25 μM) | 98 |
| Ferulic acid (25 μM) | 96 |

SORE which was enriched in FDTA-I and FDTA-II inhibited the collagenase IA activity (50%) at a very low concentration (0.04%). FDTA-I and FDTA-II acted as collagenase IA inhibitors (76% and 36% inhibition (Inh %) respectively) at 25 μM, while neither TA nor ferulic acid alone do not have any effect on this enzyme at the same concentration. It underlined the relevance of the coupling of ferulic and TA via the ester bond in the context of its anti-collagenase biological activity. Interestingly ziyuglycoside I, did not have any enzymatic inhibitory effect at 25 μM.

This result demonstrated the ability of SORE comprising FDTA-I and FDTA-II to inhibit the collagenase activity. Therefore, SORE may be used for improving the mechanical properties of the skin, particularly the ECM components, by inhibiting the activity of collagenase. Moreover, their activity on collagenase is clearly independent from the activity reported for ziyuglycoside I.

### Example 11: Anti-Hyaluronidase Inhibiting Activity of FDTA-I and FDTA-II Purified from SORE

FDTA-I and FDTA-II were purified from stimulated roots of Sanguisorba officinalis as described in example 10. In addition, ziyuglycoside I was also included in the test. For the comparison, SORE was prepared as presented in example 1.

Hyaluronidase catalyzes the degradation reaction of hyaluronic acid (HA). The test is based on the quantification of intact HA content in the presence/absence of an extract of interest and/or compounds of interest. The degradation rate of HA content is proportional to enzymatic activity of hyaluronidase and inversely proportional to the inhibition power of the extract.

The reaction mixture was prepared as follows: 37.5 μL of 20 mM PHB buffer pH 7, 187.5 μL of hyaluronidase solution (bovine hyaluronidase, Type IS, Sigma) 0.02 mg/mL, and 25 μL of sample (SORE or pure FDTA-I and FDTA-II to be tested). In the case of table 13, the pure FDTA-I and FDTA-II as well as SORE were in pure propane-1,3-diol. In the case of table 14, the pure FDTA-I and FDTA-II were in 100% ethanol. The mixture was incubated for 10 minutes at room temperature and then 250 μL of HA substrate (0.3 mg/mL in phosphate buffer) are added to initiate the reaction. Incubation is done in a Thermomixer at 37° C., under agitation (450 rpm). The reaction was monitored for 60 minutes with a sampling of the reaction mix (50 μL) every 15 minutes. These samples were inserted into the wells of a microplate containing 200 μL of BSA solution 0.1% pH 3.75 and incubated in BSA for 10 minutes. The HA formed aggregates with the BSA solution which precipitates. Thus, the amount of precipitating BSA was proportional to the optical density at 600 nm which allows to monitor the enzymatic reaction. The microplate was finally inserted into the spectrophotometer and the optical density was measured at this wavelength.

TABLE 13
Anti-hyaluronidase inhibiting activity of FDTA-I,
TA and ferulic acid at a concentration of 15
μM and SORE at a concentration of 0.01%.

| | Residual activity of hyaluronidase (%) |
|---|---|
| SORE (0.01%) | 0 |
| TA (15 μM) | 98 |
| FDTA-I (15 μM) | 9.5 |
| Ferulic acid (15 μM) | 103 |

FDTA-I acted as a hyaluronidase inhibitor at 15 μM, while neither TA nor ferulic acid alone did not have any effect on this enzyme at the same concentration. This result underlined the relevance of the coupling of ferulic and TA via the ester bond in the context of its anti-hyaluronidase biological activity.

TABLE 14
Anti-hyaluronidase inhibiting activity of Ziyuglycoside
I, FDTA-I and FDTA-II at 15 μM.

| | Residual activity of hyaluronidase (%) |
|---|---|
| FDTA-I (15 μM) | 69 |
| FDTA-II (15 μM) | 89 |
| Ziyuglycoside I (15 μM) | 107 |

FDTA-I and FDTA-II acted as hyaluronidase inhibitors at 15 μM, while ziyuglycoside I did not have any effect on this enzyme at the same concentration. This result demonstrated the ability of SORE comprising FDTA-I and FDTA-II to inhibit the hyaluronidase activity. Therefore, SORE may be used for improving the mechanical properties of the skin, particularly the ECM components, by inhibiting the activity of hyaluronidase.

### Example 12: SORE Improves Skin Elasticity and Skin Complexion

Another clinical study performed on caucasian women demonstrated the improvement of the skin elasticity measured using SEM 575® cutometer (Courage & Khazaka) which allows the determination of skin elastic properties by the calculation of the Ue parameter, well documented as being decreased with aging.

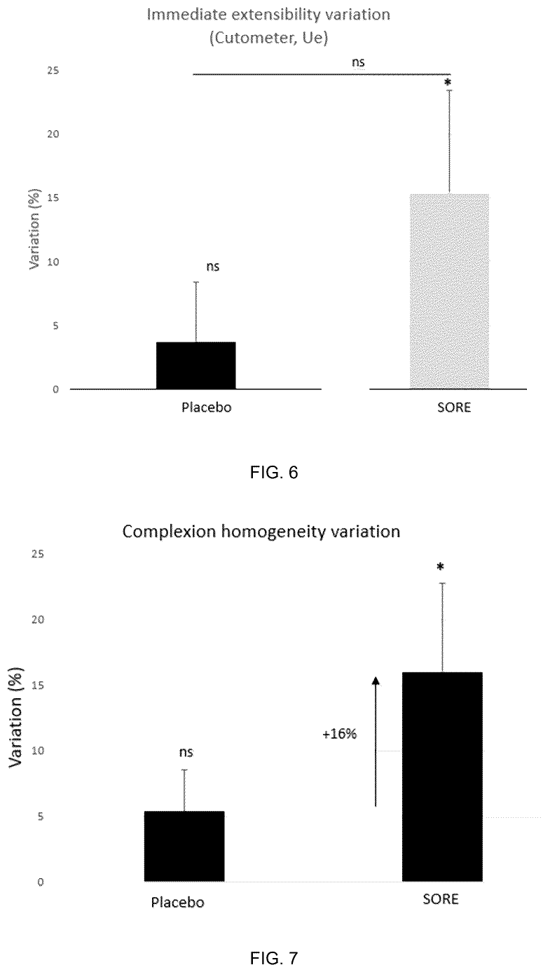

FIG. 6 shows that SORE improves skin elasticity, proven by the increase of Ue parameter after 28 days of use (+15% *: p<0.05) whereas placebo had no significant effect (+5% ns). This was consistent with the data obtained in vitro and described in the examples 10 and 11 showing that FTDA-I and FTDA-II inhibit collagenase and hyaluronidase activities known to alter skin mechanical properties such as elasticity and firmness.

The clinical study 2 also confirmed that SORE improves the skin complexion after 28 days of application. The homogeneity of face complexion was scored based on a non structured scale. Pictures were taken with Visio-Face® n°2 with the high-resolution camera Nikon D300S. Finally, the volunteers' self-evaluation of the creams was assessed through a questionnaire. FIG. 7 shows that SORE improved skin complexion which appeared more homogenous, with +16% improvement of homogeneity score (*: p<0.05) whereas placebo did not (+4% ns).

It has been demonstrated that this beneficial effect was confirmed by the volunteers themselves as more than 73% of them found their complexion more homogeneous, in contrast to the placebo (52%) after 28 days of topical application of SORE (data not shown)

### Example 13: SORE Decreases Protein Carbonylation in Corneocytes

As it is agreed that, in skin, the increase in protein's carbonylation was associated with alteration of skin quality such as skin tone or a fragile dermis, it was decided to analyse in the clinical study as described in exemple 5, also the impact of SORE on proteins carbonylation in corneocytes.

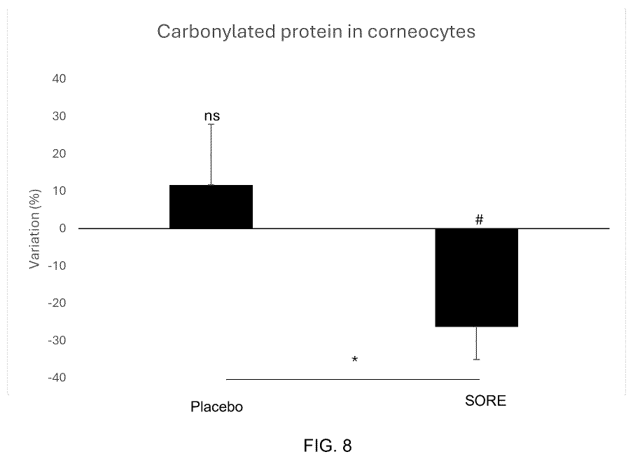

Corneocytes were collected at the surface of the skin of volunteers before (day 0) and after 28 days of use of SORE at 1% (cf. table 9) or placebo. D-squames adhesives were applied with a specific applicator (stable pressure: 50 g) for 5 to 20 seconds. First sampling was not kept, only the second one was considered. Then, corneocytes were recovered from the strips by immersion of the strip in a dissociation solution: % SDS, 20 mM DL-dithiothreitol, 5 mM EDTA, 0.1M Tris-HCl buffer pH 8, for 10 minutes at 100° C. Tubes were centrifugated. After 3 washes of 1 min at RT in dissociation solution and re-centrifugation, corneocytes were labelled with 2 mM fluorecein-5-thiosemicarbazide in 0.1M 2-morpholinoethane sulfonic acid (MES)-Na buffer (pH 5.5), for 1 h at RT. After 3 washes in phosphate buffer saline (PBS), the suspension was put onto a microscope glass slide for 12 hours. Five pictures were taken per slide. Fluorescence intensity was quantified with ImageJ software. Results shown that carbonylated proteins content was significantly decreased in corneocytes of volunteers using SORE versus placebo after 28 days of application (FIG. 8). These results demonstrated the ability of SORE to protect the skin from the deleterious effect of carbonylated proteins, and hence, to improve the skin quality including the skin tone.

1. A root extract of Sanguisorba officinalis comprising:
a tormentic acid which represents at least 1% by weight relative to the total weight of the dry extract,
a feruloylated derivative of tormentic acid having the general formula (I), 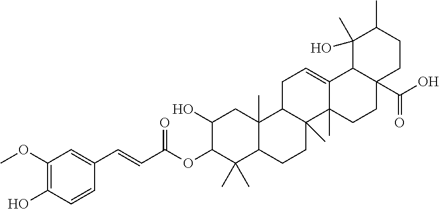
and
a feruloylated derivative of deoxy-tormentic acid having the general formula (II)

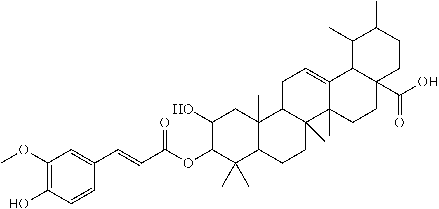

2. The root extract according to claim 1, wherein the feruloylated derivative of deoxy-tormentic acid represents at least 0.05% by weight relative to the total weight of the dry extract.

3. The root extract according to any of claim 1 or 2, wherein the feruloylated derivative of tormentic acid represents at least 0.1% by weight relative to the total weight of the dry extract.

4. The root extract according to any of claims 1 to 3, wherein the tormentic acid, the feruloylated derivative of tormentic acid and the feruloylated derivative of deoxy-tormentic acid represent together at least 1.15% by weight, relative to the total weight of the dry extract.

5. The root extract according to any of the claims 1 to 4, wherein the root extract further contains one or more ellagitanins.

6. Method for preparing a root extract of the plant Sanguisorba officinalis according to any of claims 1 to 5, the method comprising the following steps:
a) cultivating Sanguisorba officinalis under soilless conditions, in particular aeroponically,
b) stimulating the roots of said plant,
c) solid/liquid extraction by maceration of the roots obtained in step b),
d) recovering the extract obtained in step c), and
e) optionally, diluting and/or clarifying the extract recovered in step d) by successive filtrations.

7. A cosmetic or a dermatological composition comprising:
a root extract of Sanguisorba officinalis according to any of the claims 1 to 5 or a root of Sanguisorba officinalis obtained from a method according to claim 6; and
at least a cosmetically or dermatologically acceptable ingredient other than a root extract of Sanguisorba officinalis,
wherein the composition is a composition for topical use selected from the group consisting of a solution, a suspension, an emulsion, a cream, a paste, a gel, a lotion, a powder, a soap, a surfactant-containing water, an oil, a shampooing, and a spray, or wherein the composition is a nutraceutical composition which is administered orally.

8. Cosmetic use of a root extract of Sanguisorba officinalis according to any of the claims 1 to 5 as an active ingredient for skin care and/or scalp care, for promoting the light-related human being mood.

9. Cosmetic use according to claim 8, for increasing and/or stimulating the production of at least one well-being hormone in the skin cells.

10. Cosmetic use according to any of claim 8 or 9, for promoting the production of at least one well-being hormone in the skin cells.

11. Cosmetic use according to any of claims 8 to 10, for promoting or stimulating the expression of the light transducting molecules, preferably peropsins (RRH), in the skin cells

12. Cosmetic use according to any of claims 8 to 11, for maintaining or restoring the circadian rhythm of clocks proteins in skin cells.

13. Cosmetic use according to any of claims 8 to 12, for stimulating the vitamin D downstream biological pathways.

14. Cosmetic for use according to any of claim 8 or 13, for preventing or delaying the signs of photoaging on the skin, for preventing or treating the UV-related skin damages, or a combination of two or more thereof.

15. Cosmetic use according to claim 14, for decreasing and/or preventing carbonylation of proteins in skin cells.

16. Cosmetic use according to any of claim 14 or 15, for improving the mechanical properties of the skin.

17. Cosmetic use according to any of claims 14 to 16, for improving the skin elasticity, for improving the complexion of the skin, for increasing the dermis density, for maintaining or restoring the integrity of the skin cells, for maintaining or restoring the skin barrier function, for promoting the hydration of the skin, for improving the skin tone, for preventing the formation of fine lines and wrinkles, or a combination of two or more thereof.
